SEQUENCE LISTING (18 sequences)

SEQ ID NO: 1 - protein, 123 aa, Artificial Sequence
  anti-ABCB5 antibody heavy chain variable region
EVQLVESGGDLVKPGGSLKLSCAASGFTFSDYYMYWVRQTPEKRLEWVATINDGGTHTYYPDSLKGRFTISRDNAKNILYLQMSSLMSEDTAMYYCARDDYYYGSHFDAMDYWGQGTSVTVSS

SEQ ID NO: 2 - protein, 111 aa, Artificial Sequence
  anti-ABCB5 antibody light chain variable region
DIVLTQSPASLAVSLGQRATISYRASKSVSTSGYSYMHWNQQKPGQPPRLLIYLVSNLESEVPARFSGSGSGDTFTLNIHPVEEEDAATYYCQHIRELTRSEGGTKLEIKR

SEQ ID NO: 3 - protein, 5 aa, Artificial Sequence
  anti-ABCB5 antibody CDR-H1
DYYMY

SEQ ID NO: 4 - protein, 10 aa, Artificial Sequence
  anti-ABCB5 antibody CDR-H2
TINDGGTHTY

SEQ ID NO: 5 - protein, 14 aa, Artificial Sequence
  anti-ABCB5 antibody CDR-H3
DDYYYGSHFDAMDY

SEQ ID NO: 6 - protein, 15 aa, Artificial Sequence
  anti-ABCB5 antibody CDR-L1
RASKSVSTSGYSYMH

SEQ ID NO: 7 - protein, 7 aa, Artificial Sequence
  anti-ABCB5 antibody CDR-L2
LVSNLES

SEQ ID NO: 8 - protein, 8 aa, Artificial Sequence
  anti-ABCB5 antibody CDR-L3
QHIRELTR

SEQ ID NO: 9 - protein, 19 aa, Homo sapiens
KIITMFGNNDKTTLKHDAE

SEQ ID NO: 10 - protein, 19 aa, Homo sapiens
GFRFGAYLIQAGRMTPEGM

SEQ ID NO: 11 - protein, 16 aa, Homo sapiens
TGSRIGVLTQNATNMG

SEQ ID NO: 12 - protein, 51 aa, Artificial Sequence
  Synthetic peptide within Human ABCB5 aa 1200- 1250 conjugated to Keyhole Limpet Haemocyanin (KLH)
KARTGRTCLVVTHRLSAIQNADLIVVLHNGKIKEQGTHQELLRNRDIYFKL

SEQ ID NO: 13 - protein, 90 aa, Artificial Sequence
  Recombinant Protein Epitope Signature Tag (PrEST) antigen sequence
DLIVTLKDGMLAEKGAHAELMAKRGLYYSLVMSQDIKKADEQMESMTYSTERKTNSLPLHSVKSIKSDFIDKAEESTQSKEISLPEVSLL

SEQ ID NO: 14 - protein, 13 aa, Artificial Sequence
  internal sequence amino acids 460-471 of Human ABCB5 with an N-terminal Cys linker
CQTQHRNTSKKAQ

SEQ ID NO: 15 - protein, 15 aa, Homo sapiens
DKKPSIDNFSTAGYK

SEQ ID NO: 16 - protein, 15 aa, Homo sapiens
NYQNCTQSQEKLNED

SEQ ID NO: 17 - protein, 194 aa, Homo sapiens
HAFIYFAYAAGFRFGAYLIQAGRMTPEGMFIVFTAIAYGAMAIGETLVLAPEYSKAKSGAAHLFALLEKKPNIDSRSQEGKKPDTCEGNLEFREVSFFYPCRPDVFILRGLSLSIERGKTVAFVGSSGCGKSTSVQLLQRLYDPVQGQVLFDGVDAKELNVQWLRSQIAIVPQEPVLFNCSIAENIAYGDNSRV

SEQ ID NO: 18 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

(19) United States
(12) Patent Application Publication
(10) Pub. No.: US 2018/0134771 A1
(43) Pub. Date: May 17, 2018

(54) NOVEL IMMUNOMODULATORY THERAPEUTIC STRATEGIES TARGETING TUMORS IN CANCER

(71) Applicant: BioXcel Corporation, Branford, CT (US)

(72) Inventors: Krishnan NANDABALAN, Guilford, CT (US); Himani SHARMA, Balewadi (IN); Aparna Katoch SAPRA, Deonar (IN); Sanatan UPMANYU, Rajasthan (IN)

(21) Appl. No.: 15/572,422

(22) PCT Filed: May 9, 2016
(86) PCT No.: PCT/US16/31399

§ 371 Date: Nov. 7, 2017

Related U.S. Application Data
(60) Provisional application No. 62/158,408, filed on May 7, 2015.

(51) Int. Cl. C07K 16/18; A61K 39/395; A61K 45/06; A61P 35/00; A61P 35/04; A61P 35/02

(52) U.S. Cl. CPC C07K 16/18; A61K 39/39533; A61K 45/06; A61K 2039/505; A61P 35/04; A61P 35/02; A61P 35/00

(57) ABSTRACT

The present invention discloses a method of treating, preventing or ameliorating tumor growth by immune response modulation via targeting ABCB5 and an immune checkpoint molecule related pathways using various therapeutic agents such as antibody or small molecule. The present invention also provides use of an ABCB5 inhibitor and an immune checkpoint inhibitor(s) for enhancing, increasing, promoting, expressing, modulating desirable immune response for prevention and treatment of tumors.

### CROSS-REFERENCE TO RELATED APPLICATIONS

This application claims priority to U.S Provisional Application Ser. No. 62/158,408, filed May 7, 2015, which is hereby incorporated by reference in its entirety.

### FIELD OF THE INVENTION

The present invention relates to combining immunomodulatory approaches for the treatment of cancer. More specifically the use of antibodies or small molecules that bind and target both ABCB5 and an immune checkpoint molecule for the treatment of cancer.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: BIOX_003 01WO_SeqList_ST25.txt, date recorded: May 4, 2016, file size 11 kilobytes).

### BACKGROUND OF THE INVENTION

Immunotherapy for the treatment of cancer has evolved alongside our improved understanding of immune system. In particular, an appreciation of the ability of cancer cells to subvert the antitumor immune response has provided a rationale for the development of novel immunotherapies that target immune checkpoints responsible for affecting tumor microenvironment. Cells and molecules of the immune system are the fundamental components of the tumor microenvironment. Tumor microenvironment contributes to tumor initiation, tumor progression and responses to therapy.

ABCB5 (ATP binding cassette sub-family B member 5), also known as P-glycoprotein ABCB5 is located in the plasma membrane, with five transmembrane helices flanked by both extracellular and intracellular ATP-binding domains. It has two isoforms known as ABCB5 alpha and ABCB5 beta. It belongs to the ATP-binding cassette (ABC) transporter superfamily of integral membrane proteins. It is expressed in many different tissues, including brain, intestine, kidney, mammary gland, testis and skin. It is highly expressed in malignant melanomas than in benign melanomas.

Brian J. Wilson et al., Cancer Research, 74(15): 4196-4207 discloses that ABCB5 plays a role in cancer stem cell maintenance and tumor growth. However, the present invention provides an entirely different concept to use an anti-ABCB5 antibody in order to induce immune stimulation in combination with an immune checkpoint inhibitor(s) (such as PD-1 antagonist, PD-L1 antagonist, PD-L2 antagonist and CTLA4 antagonist) for the treatment of cancer.

U.S. Pat. No. 7,928,202 assigned to The Brigham and Women's Hospital Inc. discloses an isolated antibody that binds to ABCB5 and the use of said anti-ABCB5 antibody in conjugation to a therapeutic agent for the treatment of cancer. However, it does not disclose the combination of anti-ABCB5 antibody with immune checkpoint inhibitors (such as PD-1 antagonist, PD-L1 antagonist, PD-L2 antagonist and CTLA4 antagonist) as an effective therapy for tumor evasion.

PCT Publication No. WO2010065711 assigned to Adimab Inc. discloses anti-ABCB5 antibody or use of anti-ABCB5 antibody alone or in combination with second agent such as camptothecin or mitoxantrone for the treatment of cancer. However, it is silent about the use of immune checkpoint inhibitors in combination with anti-ABCB5 antibody that leads to a promising immunotherapy for cancer patients.

Immune checkpoint molecules such as programmed cell death 1 (PD-1) is a cell surface signalling receptor that plays a critical role in the regulation of T-cell activation and tolerance. PD-1 is primarily expressed on activated T cells, B cells, and myeloid cells. PD-1 is highly expressed on tumor-infiltrating lymphocytes, and its ligands such as PD-L1 are up-regulated on the cell surface of many different tumors. It has been shown that inhibition of the PD-1/PD-L1 interaction mediates potent antitumor activity in preclinical models (U.S. Pat. Nos. 8,008,449 and 7,943,743). Another molecule, cytotoxic T-lymphocyte antigen 4 (CTLA-4) acts as an immune checkpoint, in downregulating the immune system. It is found on the surface of T-cells and is involved in the maintenance of T cell homeostasis. U.S. Pat. No. 7,452,535 discloses a method of treating cancer by administration of anti-CTLA4 antibodies. These patents do not provide any teachings to control the tumor growth by modulating the immune response via targeting both ABCB5 and an immune checkpoint molecule. However, these immune checkpoint inhibitors possess dose associated toxicities and a few segment of cancers are not responsive to these checkpoint inhibitors when used alone. Hence, the inventors of the present invention have come up with a promising approach to evade the tumor cells by administration of an ABCB5 inhibitor with an immune checkpoint inhibitor that may enhance or prolong the anti-tumor and immunomodulatory effects of the immune checkpoint inhibitor, enable a subject to respond to an immune checkpoint inhibitor, or enable the reduction of the toxicity or the dose of an immune checkpoint inhibitor.

The present invention relates to therapeutic agent(s) with specifically binds and targets ABCB5 and immune checkpoint molecule(s) such as PD1-axis or CTLA4, and use of such therapeutic agent(s) in combination therapy for the treatment of cancer. In particular, the present invention provides combination of one or more antibodies that specifically bind and target ABCB5 and immune checkpoint molecule such as PD1-axis or CTLA4, and uses thereof.

Therefore, current invention's focus is to design therapies for such individual targets which may be targeted either separately or synergistically for an improved therapeutic response.

The present inventors have also identified a method of targeting novel combinations of targets that can lead to mitigation of tumor growth.

Despite advances in the field, however, there remains a need for improved methods and compositions for treating cancer or tumor.

### SUMMARY OF THE INVENTION

The present invention provides use of a therapeutic agent as an ABCB5 inhibitor, more specifically an antibody for control of tumor growth by modulating the immune response via targeting ABCB5. This ABCB5 antibody works by activating an immune response that will help in delaying the growth of human tumors.

The present inventors discovered that the programmed death 1 (PD-1) receptor as well as its ligands (PD-L1/2) that exhibit tumor-induced immune suppression when intervened together with the depletion of self-renewing cancer cells as in case of the ABCB5+ cell populations in cancer holds potential advantage to be explored in immuno-therapies. An ABCB5 inhibitor has a dual mechanism, firstly it acts as a functional inhibitor and causes T cell activation via IL-2 production and also act as a depleting antibody by eliminating the ABCB5+ cancer stem cells. Therefore, the tumor metastasis or growth could be effectively controlled by modulating the immune response via targeting the ABCB5 and immune checkpoint molecule such as PD-1, PD-L1, PD-L2 or CTLA4 by using combination of an ABCB5 inhibitor (for example, anti-ABCB5 antibody) and an immune checkpoint inhibitor.

It is the principal object of the present invention to provide a method of treating tumors associated with the increased levels of ABCB5 and/or immune checkpoint molecule(s). It also provides methods of treating tumors by modulating the immune response via targeting ABCB5 and immune checkpoint molecule(s). The present disclosure also provides method of treating tumors having ABCB5 protein expressed at normal levels, as determined by the expression level on non-tumor tissue.

In yet another aspect, the present invention provides a method of enhancing, increasing, promoting, expressing, modulating desirable immune response in a subject, comprising administering an effective amount of at least one therapeutic agent targeting tumors associated with increased levels of ABCB5 and/or an immune checkpoint molecule(s) in an amount to enhance, increase, promote, express, modulate immune response in the subject, wherein the subject has been diagnosed for tumor.

The therapeutic agent may be an antibody (including monoclonal, polyclonal or nanobody) or small molecule.

In yet another aspect, the present invention provides the use of at least one therapeutic agent which includes antibody or small molecule targeting both ABCB5 and an immune checkpoint molecule(s) for the treatment or prevention of tumors.

In another aspect, the present invention provides a pharmaceutical composition comprising
(a) an effective amount of at least one therapeutic agent that binds and targets both ABCB5 and an immune checkpoint molecule(s);
(b) an optional anti-tumor agent and
(c) one or more pharmaceutically acceptable carriers or adjuvants
wherein administering the composition to a subject having a tumor treats, prevents or delays tumor growth or metastasis in the subject.

The therapeutic agent which binds and targets an ABCB5, is an ABCB5 inhibitor and the therapeutic agent which binds and targets an immune checkpoint molecule, is an immune checkpoint inhibitor.

In one another aspect, the present invention provides a pharmaceutical composition comprising
(a) an effective amount of an ABCB5 inhibitor(s);
(b) an effective amount of an immune checkpoint inhibitor(s);
(c) an optional anti-tumor agent and
(d) one or more pharmaceutically acceptable carriers and/or adjuvants
wherein administering the composition to a subject having a tumor treats, prevents or delays tumor growth or metastasis in the subject.

Examples of immune checkpoint inhibitors, include but are not limited to PD-1 antagonist, PD-L1 antagonist, PD-L2 antagonist, CTLA4 antagonist and combination thereof.

Examples of ABCB5 inhibitors, include but are not limited to anti-ABCB5 antibody (including anti-ABCB5 nanobody) or small molecules targeting ABCB5 and the preferred one is anti-ABCB5 antibody which is a monoclonal that binds and targets the amino acid residues corresponding to 481-674 of the human ABCB5.

In another aspect, the present invention provides a method of treating, delaying or preventing the metastasis of tumor in a subject, comprising administering an effective amount of at least one therapeutic agent that binds and targets both ABCB5 and PD-1 axis, wherein the subject has been diagnosed for tumor associated with the increased levels of ABCB5 and/or PD-1 axis.

In yet another aspect, the present invention provides a method of treating, delaying or preventing the metastasis of tumor in a subject, comprising administering an effective amount of at least one therapeutic agent that binds and targets both ABCB5 and CTLA4, wherein the subject has been diagnosed for tumor associated with the increased levels of ABCB5 and/or CTLA4.

In some aspects, the present invention provides a therapeutic agent that binds and targets ABCB5 for use in the treatment of a tumor ameliorated by stimulation of an immune response, wherein in said treatment an immune checkpoint inhibitor, is co-administered.

In some aspects, the present invention provides a pharmaceutical composition for use in combination with an immune checkpoint inhibitor comprising PD-1 antagonist, PD-L1 antagonist, PD-L2 antagonist and CTLA4 antagonist for treating a tumor, wherein said pharmaceutical composition comprises an ABCB5 inhibitor with one or more pharmaceutically acceptable carrier(s) or adjuvant(s).

In some aspects, the present invention provides a kit comprising:
(a) a first composition comprising an ABCB5 inhibitor and
(b) a second composition comprising an immune checkpoint inhibitor.

In some aspects, the present invention provides a combination therapy for the treatment of tumor, the said combination comprises:
(a) a therapeutic agent that binds and targets ABCB5 and
(b) an immune checkpoint inhibitor.

In yet another embodiment, the present invention provides a method of identification of upregulation of ABCB5 and an immune checkpoint molecule(s) activity in cancer patients and treating them using combination therapy of the present invention.

All publications, patent applications, patents, and other references mentioned herein are incorporated by reference in their entirety.

Other features, objects, and advantages of the invention will be apparent from the description and drawings, and from the claims.

### BRIEF DESCRIPTION OF THE DRAWINGS

FIG. 1. shows the dose dependent effect of anti-ABCB5 antibody (Novus; Cat. No. NBP2-22213) in the increase of IL2 production in the presence of a fixed flat concentration of the PD-1 antagonist 200 ng/ml (BPS Biosciences, Cat. No. 71120) in the mixed cultures of hPBMCs and ABCB5+ (WM-2664) melanoma cell line.

FIG. 2. shows that the combination of anti-ABCB5 antibody (Novus; Cat. No. NBP2-22213) and the immune check point inhibitor, PD-1 antagonist (BPS Biosciences, Cat. No. 71120) caused a synergistic three-fold increase in the release of IL-2 from the effector hPBMCs in the presence of the ABCB5+ (WM-2664) melanoma cell line.

### DETAILED DESCRIPTION OF THE INVENTION

### Abbreviations:

As used herein, the following abbreviations have the following meanings:
ABCB5: ATP binding cassette sub-family B member 5
ATCC: American Type Culture Collection
ADCC (Antibody dependent cellular cytotoxicity)
Ab: Antibody
A2AR: A2A adenosine receptor
APC: Antigen presenting cell
BTLA: B- and T-lymphocyte attenuator
B7-H1: B7 homolog 1
CD: Cluster of differentiation
CTLA4: Cytotoxic T-lymphocyte associated protein 4
CDR: Complementarity determining region
CHK: Checkpoint kinase
ELISA: Enzyme-linked immunosorbent assay
FBS: Fetal bovine serum
IL: Interleukin
LPS: Lipopolysaccharide
LAG3: Lymphocyte activation gene 3 protein
KIR: Killer immunoglobulin receptor
MMP: Matrix metalloproteinases
RPMI: Roswell Park Memorial Institute medium
PHA: Phytohaemagglutinin
PBS: Phosphate buffer saline
PD1: Programmed Cell Death 1
PBMC: Peripheral blood mononuclear cell
TIM3: T-cell immunoglobulin and mucin-domain containing-3
VISTA: V domain-containing Ig suppressor of T-cell activation

Immune modulation in cancer refers to a range of treatments aimed at harnessing a patient's immune system to achieve tumor control, stabilization, and potential eradication of disease.

During immune surveillance, the host provides defense against foreign antigens, while ensuring its limited activation against self-antigens. Immune checkpoints are cell surface molecules that serve as endogenous regulators of the immune response, limiting autoimmunity by mediating co-inhibitory signaling pathways. In cancer, these pathways are important in the tumor microenvironment and draining lymph nodes, leading to a state of T-cell exhaustion, thereby allowing tumor escape from immune surveillance, and unchecked tumor growth.

The present invention provides that the immune checkpoint molecule could be targeted using various targeting agents either by antagonizing co-inhibitory immunologic pathways or activating co-stimulatory pathways. These immune checkpoint targeting agents (include inhibitors) are clinically active in a variety of malignancies, including those not traditionally classified as immunogenic.

One of the targets is ABCB5, employed in the present therapeutic approach for treating cancer in combination with an immune checkpoint molecule(s) (for example, PD-1, PD-L2, PD-L1 or CTLA4) or as an adjunctive therapy to sensitize cancer cells to chemotherapeutic agents, especially in those patients with currently refractory metastatic disease.

ABCB5 is an established drug efflux transporter known to lead to chemo-resistance. It has also been identified to be expressed in malignant melanoma initiating cells aiding in the existence of cancer stem cells. ABCB5 controls secretion of IL-1 beta which activates IL-8.

A number of distinct forms of ABCB5 have been shown to be expressed in various tissue types, including, but not limited to, melanocytes, melanoma cells, testis, mammary tissue, and retinal pigmented epithelium. ABCB5 has also been found to be expressed at the transcriptional level in a number of cancer subtypes, including malignant melanoma, breast cancer, colorectal cancer and hepatocellular carcinoma and also has been linked to leukemia.

Since many of the immune checkpoint molecules are also regulated by interactions between specific receptor and ligand pairs, antibodies or other agents can be used to block this interaction and prevent immunosuppression. The two checkpoint receptors that have received the most attention in recent years are CTLA-4 and PD-1 axis. CTLA-4, PD-1 and its ligands are members of the CD28-B7 family of co-signaling molecules that play important roles throughout all stages of T-cell function and other cell functions. The PD-1 receptor is expressed on the surface of activated T cells (and B cells) and, under normal circumstances, binds to its ligands (PD-L1 and PD-L2) that are expressed on the surface of antigen-presenting cells, such as dendritic cells or macrophages. This interaction sends a signal into the T cell and essentially switches it off or inhibits it. Cancer cells take advantage of this system by driving high levels of expression of PD-L1 on their surface. This allows them to gain control of the PD-1 pathway and switch off T cells expressing PD-1 that may enter the tumor microenvironment, thus suppressing the anticancer immune response.

A first-in-class immunotherapy, ipilimumab (Yervoy®), a monoclonal antibody that targets cytotoxic T-lymphocyte-associated antigen 4 (CTLA-4) on the surface of T cells, was approved for the treatment of melanoma. Now, a new targeted immunotherapy aimed at the programmed death-1 (PD-1) T-cell receptor or its ligand (PD-L1 or PD-L2) may also prove to be effective.

Various cancers, such as melanoma, hepatocellular carcinoma, glioblastoma, lung, kidney, breast, ovarian, pancreatic, and esophageal cancers, as well as hematological malignancies, have positive PD-L1 expression, and this expression has been correlated with poor prognosis.

PD-L1 expression in tumors has been associated with poor prognosis in many tumor types, which has been interpreted as consistent with its role in immune evasion. However, recent reports have challenged this notion to some extent, documenting favorable outcomes in melanoma patients with PD-L1 positive tumors. PD-L1 expression in the tumors was co-localized with tumor T cell infiltration and interferon-γ mRNA expression, suggesting an “adaptive resistance” mechanism in which PD-L1 expression is a reflection for the melanoma being actively attacked by presumably melanoma-specific T cells, explaining the improved prognosis.

Therefore, the clinical efficacy seen with PD-1/PDL1 pathway blockade in patients with multiple different tumor types, most of whom were heavily pre-treated, suggests that the PD-1 pathway is an important target that many tumors may utilize to evade destruction by the host immune response. This observation in conjunction with the favorable toxicity profile of PD-1 inhibition indicates potential broad applicability in patients with advanced tumors. Even more meaningful may be the durability of tumor responses observed with PD-1/PDL1 pathway inhibition, which has reached the 10-year mark for some melanoma patients who have not required any treatment for many years. Currently, at least seven checkpoint inhibitor agents are in clinical trials. Among them are monoclonal anti-PD-1 antibodies, both fully human and humanized, as well as a fully human anti-PD-L1 antibody and a fusion protein combining the extracellular domain of PD-L2 and IgG1. Each of these agents is designed to block the interaction between PD-1 and its ligands, and thus keep the T-cell (or other cell) on/off switch in the “on” position, although each one of them have slightly different mechanisms of action.

The upregulation of PD-L1 is a common phenomenon in leukemia, lymphomas and other associated cancers that leads to double T-cell immunodeficiency, low proliferation and activation effects, and higher immune suppression in patients.

An advantage of combination of ABCB5 and an immune checkpoint molecule targeted therapeutic approaches is that they could regulate both active tumor growth as well as are directed at tumorigenic stem cells, whereas conventional therapeutics target only the bulk population of tumor cells.

Firstly, detected in tissues derived from the neuro-ectodermal lineage including melanocyte progenitors, melanoma cell lines and patient specimens, ABCB5 expression is found to be associated with self-renewal, differentiation and tumorigenic abilities. Cumulative data till date including those from melanoma xenografts mouse model as well as clinical biopsies have indicated the abundance of ABCB5+ cells in clinical melanoma specimens that correlates positively with the neoplastic progression. Moreover, as a member of the ABC transporter family, ABCB5 is thought to play a role in drug efflux as supported by experiments that showed the intracellular accumulation of rhodamine 123 or doxorubicin in melanoma and hepatocellular carcinoma cells. The ABCB5+ melanoma cells have also displayed T cell inhibition, in terms of IL-2 release and have also shown survival advantage in case of chemotherapies associated with melanoma. In this context, the programmed death 1 (PD-1) receptor as well its ligands (PD-L1/2) as well as CTLA4 that exhibit tumor-induced immune suppression when intervened together with the inhibition of self-renewing cancer cells as in case of the ABCB5+ cell populations in cancers holds potential advantage to be explored in immuno-therapies. Therefore, in the current invention, this combination strategy has shown the therapeutic advantage as indicated by the release of IL-2 from human PBMCs in the presence of ABCB5+ cell line where both anti-ABCB5 antibody as well as PD-1 antagonist/CTLA4 antagonist were available in the mixed culture milieu, as compared to either of the agents alone.

The present invention relates to a combination of an ABCB5 inhibitor and an immune checkpoint inhibitor to promote an effective anti-tumor response. The details of the various features of the present invention are as follows:

Various therapeutic agents/antibodies of the present invention are described below:

### I. Therapeutic Agents

A therapeutic agent that binds and targets ABCB5 is an ABCB5 inhibitor, which includes anti-ABCB5 antibody (including- anti-ABCB5 nanobody) or small molecule targeting ABCB5. The preferred therapeutic agent is anti-ABCB5 antibody.

### (a) Anti-ABCB5 Antibodies

One or more antibodies that bind and targets ABCB5 may include newly invented antibody targeted to ABCB5 or well-known antibody. Any anti-ABCB5 antibodies known in the art and described herein may be used in the methods.

The anti-ABCB5 antibody is able to bind and target ABCB5. According to the invention, the antibody may be a human antibody, a humanized antibody, bi-specific antibody or a chimeric antibody. Moreover, the antibody may consist of Fab, Fab′2, scFv, SMIP, affibody, avimer, nanobody or “domain antibody”. One of the anti-ABCB5 antibodies of the present invention includes mAb 3C2-1 D12. Anti-ABCB5 antibodies also include recombinant monoclonal antibody.

The term “antibody” as used herein is meant in a broad sense and includes immunoglobulin molecules including polyclonal antibodies, monoclonal antibodies including murine, human, human-adapted, humanized and chimeric synthetic, recombinant, hybrid, mutated, engineered, grafted antibodies, antibody fragments, monospecific, bispecific or multi-specific antibodies, dimeric, tetrameric or multimeric antibodies, nanobody, single chain antibodies and antibody drug conjugate. The antibodies also include recombinant monoclonal antibody. As used herein, unless otherwise indicated, “antibody fragment” or “antigen binding fragment” refers to antigen binding fragments of antibodies, i.e. antibody fragments that retain the ability to bind specifically to the antigen bound by the full-length antibody, e.g. fragments that retain one or more CDR regions. Examples of antibody binding fragments include, but are not limited to, Fab, F(ab′)₂, Fv, scFv, bi-scFv, bi-Ab, Fd, dAb, and other antibody fragments that retain antigen-binding function, i.e., the ability to bind ABCB5 specifically; diabodies; linear antibodies; single-chain antibody molecules, e.g., sc-Fv; nanobodies and multispecific antibodies formed from antibody fragments.

An anti-ABCB5 antibody is disclosed in U.S. Pat. No. 7,928,202 and assigned to The Brigham and Women's Hospital, Inc. U.S. Pat. No. 7,928,202 discloses an isolated peptide that selectively binds to ABCB5 comprises of the immunoglobulin heavy chain variable domain, wherein: (i) CDR1-H1 comprises an amino acid sequence of SEQ ID NO. 3; (ii) CDR2-H2 comprises an amino acid sequence of SEQ ID NO. 4; and (iii) a CDR3-H3 sequence comprises an amino acid sequence of SEQ ID NO. 5. It further comprises of a light chain variable domain wherein CDR1-L1 has an amino acid sequence of SEQ ID NO. 6, a CDR2-L2 that has an amino acid sequence of SEQ ID NO. 7 and/or a CDR3-L3 that has an amino acid sequence of SEQ ID NO. 8. The isolated peptide may bind to human ABCB5 and may be an antibody. The isolated peptide comprises of a monoclonal antibody having a heavy chain variable region having an amino acid sequence of SEQ ID NO: 1 and a light chain variable region having an amino acid sequence of SEQ ID NO:2.

SEQ ID NO: 1
HC-F1 CDR-H1
EVQLVESGGDLVKPGGSLKLSCAASGFTFS DYYMY
HC-F2 CDR-H2
WVRQTPEKRLEWVA TINDGGTHTY
HC-F3
YPDSLKGRFTISRDNAKNILYLQMSSLMSEDTAMYYCAR
CDR-H3 HC-F4
DDYYYGSHFDAMDY WGQGTSVTVSS
SEQ ID NO: 2
LC-F1 CDR-L1
DIVLTQSPASLAVSLGQRATISY RASKSVSTSGYSYMH
LC-F2 CDR-L2
WNQQKPGQPPRLLIY LVSNLES
LC-F3 CDR-L3
EVPARFSGSGSGDTFTLNIHPVEEEDAATYYC QHIRELTR
LC-F4
SEGGTKLEIKR
CDR-H1, CDR-H2 and CDR-H3 Sequences:
CDR-H1:
SEQ ID NO: 3
DYYMY
CDR-H2:
SEQ ID NO: 4
TINDGGTHTY
CDR-H3:
SEQ ID NO: 5
DDYYYGSHFDAMDY
CDR-L1, CDR-L2 and CDR-L3 Sequences:
CDR-L1:
SEQ ID NO: 6
RASKSVSTSGYSYMH
CDR-L2:
SEQ ID NO: 7
LVSNLES
CDR-L3:
SEQ ID NO: 8
QHIRELTR

WO2010065711 discloses an isolated polypeptide consisting essentially of sequence of amino acids of residues 273-288, 275-284, 265-288, and 273-283.

Anti-ABCB5 antibody includes antibodies which are raised in mouse against epitope corresponding to amino acids region 481-674 of human ABCB5 (SEQ ID NO:17); or raised in goat against epitope corresponding to amino acids region 88-102, 460-471, 364-378 and 905-916 of human ABCB5; or raised in rabbit against epitope corresponding to amino acids region 150-200, 450-500, 497-551, 563-812, 587-790, 1200-1250, and 1-30 amino acid of N-terminal region of human ABCB5; or raised in sheep against epitope corresponding to Ile141-Val247 of human ABCB5. Anti-ABCB5 antibody include antibodies which are epitope corresponding to extracellular loop of amino acids regions 273-288, 275-284, 265-288, 273-283, 312-382 and 491-508 of C-terminal of ABCB5.

Preferably, anti-ABCB5 antibody is a monoclonal antibody which is raised in mouse against epitope corresponding to amino acids residues corresponding to 481-674 of human ABCB5 and procured from Novus which binds and targets a part of the ABC membrane domain of the ABCB5 beta variant which is expressed in melanoma stem cells, normal melanocytes, and other types of pigment cells.

Anti-ABCB5 antibody include antibody which are raised in rabbit having the antigen sequence as follows:

| Antigen Sequence | SEQ ID NO: | Vendor |
|---|---|---|
| KARTGRTCLVVTHRLSAI | SEQ ID NO: 12 | www.abcam.com/abcb5- |
| QNADLIVVLHNGKIKEQG | | antibody-ab203120.html |
| THQELLRNRDIYFKL | | |
| DLIVTLKDGMLAEKGAH | SEQ ID NO: 13 | www.sigmaaldrich.com/catalog/ |
| AELMAKRGLYYSLVMSQ | | product/sigma/hpa026975? |
| DIKKADEQMESMTYSTE | | 1ang = en&region = US |
| RKTNSLPLHSVKSIKSDFI | | https://atlasantibodies.com/#!/ |
| DKAEESTQSKEISLPEVSL | | products/ABCB5-antibody- |
| L | | HPA026975 |

Anti-ABCB5 antibody include antibody which are raised in goat having the sequence as follows:

| Antigen Sequence | SEQ ID NO: | Vendor |
|---|---|---|
| C-QTQHRNTSKKAQ | SEQ ID NO: 14 | www.abcam.com/abcb5- |
| | | antibody-ab77549.html |
| DKKPSIDNFSTAGYK | SEQ ID NO: 15 | www.abcam.com/abcb5- |
| | | antibody-ab126864.html |
| NYQNCTQSQEKLNED | SEQ ID NO: 16 | http://www.abcore- |
| | | inc.com/anti-abcb5-antibody- |
| | | p-ac11-0408 |

An anti-ABCB5 antibody may bind to an epitope on human ABCB5 extracellular domain (SEQ tD NOS:9₂ 10, 11)

Domain 1:
(SEQ ID: 9)
KIITMFGNNDKTTLKHDAE
Domain 2:
(SEQ ID: 10)
GFRFGAYLIQAGRMTPEGM
Domain 3:
(SEQ ID: 11)
TGSRIGVLTQNATNMG

In other aspects, the anti-ABCB5 antibody may be a nanobody. Nanobody technology was developed from the discovery that antibodies from camels and llamas (Camelidae, camelids) have heavy chains but no light chains. The antigen-binding site of such antibodies is one single domain, and may be referred to as VHH. See, e.g., U.S. Pat. Nos. 5,800,988 and 6,005,079 and International Application Publication Nos. WO 94/04678, WO 94/25591 and EP 2673297 which are incorporated by reference.

The anti-ABCB5 antibodies can be procured from R&D systems, Thermofisher, Rockland immunochemicals, Amsbio LLC, Creative Diagnostics, Santa Cruz Biotechnology, Inc., EMD Millipore, OriGene Technologies, Atlas Antibodies, United States Biological, antibodies-online, Raybiotech, Inc., GenWay Biotech, Inc., Abnova Corporation, Abcamm Source BioScience, Bioss Inc., Abbexa Ltd, ProSci, Inc., LifeSpan BioSciences, Novus Biologicals, LifeSpan BioSciences, Biorbyt and so on.

### (b) Immune Checkpoint Inhibitors

Immune checkpoint inhibitors include PD1 antagonist, PD-L1 antagonist, PD-L2 antagonist CTLA4 antagonist, VISTA antagonist, TIM3 antagonist, LAG3 antagonist, OX40 agonist, IDO antagonist, KIR2D antagonist, A2AR antagonist and the preferred one is PD1 axis antagonist, CTLA4 antagonist or combination thereof.

### PD1 Axis Antagonists

PD1 axis antagonists include PD1 antagonist (for example anti-PD-1 antibody), PD-L1 antagonist (for example anti-PD-L1 antibody) and PD-L2 antagonist (for example anti-PD-L2 antibody).

As used herein, the terms “Programmed Death 1,” “Programmed Cell Death 1,” “Protein PD-1,” “PD-1,” PD1,” “PDCD1,” “hPD-1” and “hPD-I” are used interchangeably, and include variants, isoforms, species homologs of human PD-1, and analogs having at least one common epitope with human PD-1. The complete human PD-1 sequence can be found under GenBank Accession No. U64863. In particular aspects, the PD-1 antagonist binds the PD-1 protein of SEQ ID NO:18 (uniprot ID Q15116).

As used herein, the terms “Programmed Cell Death 1 Ligand 1”, “PD-L1”, “PDL1”, “PDCD1L1”, “PDCD1LG1”, “CD274”, “B7 homolog 1”, “B7-H1”, “B7-H”, and “B7H1” are used interchangeably, and include variants, isoforms, species homologs of human PDL-1, and analogues having at least one common epitope with human PDL-1.

The protein programmed death 1 (PD-1) is an inhibitory member of the CD28 family of receptors, that also includes CD28, CTLA-4, ICOS and BTLA.

Two ligands for PD-1 have been identified, PD-L1 and PD-L2, that have been shown to downregulate T cell activation upon binding to PD-1 (Freeman et al. (2000) J Exp. Med. 192: 1027-34; Latchman et al. (2001) Nat Immunol. 2:261-8; Carter et al. (2002) Eur. J Immunol 32:634-43). Both PD-L1 and PD-L2 are B7 homologs that bind to PD-1, but do not bind to other CD28 family members. PD-L1 is abundant in a variety of human cancers (Dong et al. (2002) Nat. Med. 8:787-9). The interaction between PD-1 and PD-L1 results in a decrease in tumor infiltrating lymphocytes, a decrease in T-cell receptor mediated proliferation, and immune evasion by the cancerous cells (Dong et al. (2003) J. Mol. Med. 81:281-7; Blank et al. (2005) Cancer Immunol. Immunother. 54:307-314; Kenosha et al. (2004) Clin. Cancer Res. 10:5094-100). Immune suppression can be reversed by inhibiting the local interaction of PD-1 with PD-L1, and the effect is additive when the interaction of PD-1 with PD-L2 is blocked as well (Iwai et al. (2002) Proc. Nat'l. Acad. Sci. USA 99:12293-7; Brown et al. (2003) J. Immunol. 170: 1257-66).

The methods of the present invention involve the use of a PD-1 antagonist (e.g., an antibody) in combination with anti-ABCB5 antibody for treating tumor or cancer. Accordingly, PD-1 antagonists of the invention bind to ligands of PD-1 and interfere with, reduce, or inhibit the binding of one or more ligands to the PD-1 receptor, or bind directly to the PD-1 receptor, without engaging in signal transduction through the PD-1 receptor. In one embodiment, the PD-1 antagonist binds directly to PD-1 and blocks PD-1 inhibitory signal transduction. In another embodiment the PD-1 antagonist binds to one or more ligands of PD-1 (e.g., PD-L1 and PD-L2) and reduces or inhibits the ligand(s) from triggering inhibitory signal transduction through the PD-1. In one embodiment, the PD-1 antagonist binds directly to PD-L1, inhibiting or preventing PD-L1 from binding to PD-1, thereby blocking PD-1 inhibitory signal transduction.

PD-1 antagonists used in the methods and compositions of the present invention include PD-1 binding scaffold proteins and include, but are not limited to, PD-1 ligands, antibodies and multivalent agents. In a particular embodiment, the antagonist is a fusion protein, such as AMP-224. In another embodiment, the antagonist is an anti-PD-1 antibody (“PD-1 antibody”). Anti-human-PD-1 antibodies (or VH and/or VL domains derived therefrom) suitable for use in the invention can be generated using methods well known in the art.

In some embodiment, the antibodies interfering with PD-1 is an anti-PD-1 antibody (e.g., a human antibody, a humanized antibody, or a chimeric antibody). In some embodiments, the anti-PD-1 antibody is selected from the group consisting of MDX-1106 (also known as nivolumab, MDX-1106-04, ONO-4538, BMS-936558, and Opdivo®), Merck 3475 (also known as pembrolizumab, MK-3475, lambrolizumab, Keytruda®, and SCH-900475), and CT-011 (also known as pidilizumab, hBAT, and hBAT-1). In some embodiments, the PD-1 binding antagonist is AMP-224 (also known as B7-DCIg). In some embodiments, the anti-PD-L1 antibody is selected from the group consisting of YW243.55.S70, MPDL3280A, MDX-1105, and MEDI4736. MDX-1105, also known as BMS-936559, is an anti-PD-L1 antibody described in WO2007/005874. Antibody YW243.55. S70 is an anti-PD-L1 described in WO 2010/077634 A1. MEDI4736 is an anti-PD-L1 antibody described in WO2011/066389 and US2013/034559. MDX-1106, also known as MDX-1106-04, ONO-4538 or BMS-936558, is an anti-PD-1 antibody described in U.S. Pat. No. 8,008,449 and WO2006/121168. Merck 3745, also known as MK-3475 or SCH-900475, is an anti-PD-1 antibody described in U.S. Pat. No. 8 345 509 and WO2009/114335. CT-011 (Pidizilumab), also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342. Atezolimumab is an anti-PD-L1 antibody described in U.S. Pat. No. 8,217,149. Avelumab is an anti-PD-L1 antibody described in US 20140341917. CA-170 is a PD-1 antagonist described in WO2015033301 & WO2015033299. Other anti-PD1 antibodies are disclosed in U.S. Pat. No. 8,609,089, US 2010028330, and/or US 20120114649.

In some embodiments, the anti-PD-1 antibody is MDX-1106. Alternative names for “MDX-1106” include MDX-1106-04, ONO-4538, BMS-936558 or nivolumab. In some embodiments, the anti-PD-1 antibody is Nivolumab (CAS Registry Number: 946414-94-4).

In some embodiments, the anti PD-L2 antibody is AMP-224 or rHIgM12B7.

In one embodiment, the PD-1 inhibitor is an anti-PD-1 antibody chosen from Nivolumab, Pembrolizurnab or Pidilizumab.

Examples of anti-PD-L1 antibodies useful for the methods of this invention, and methods for making thereof are described in PCT patent application WO 2010/077634 A1, which is incorporated herein by reference.

The anti-PD-L1 antibodies useful in this invention, including compositions containing such antibodies, such as those described in WO 2010/077634 A1 and U.S. Pat. No. 8,217,149, may be used in combination with an ABCB5 inhibitor to treat cancer.

The antibody or antigen binding fragment thereof, may be made using methods known in the art, for example, by a process comprising culturing a host cell containing nucleic acid encoding any of the previously described anti-PD-L1, anti-PD-1, or anti-PD-L2 antibodies or antigen-binding fragment in a form suitable for expression, under conditions suitable to produce such antibody or fragment, and recovering the antibody or fragment.

With regard to anti-PD-1 antibodies, these are known and include nivolumab and lambrolizumab, AMP-224, MDPL3280A, MEDI4736 and MSB0010718C. Anti-PD-1 antibody may be procured from BPS Biosciences and Bio X cell.

In one embodiment, PD-1 antagonist is selected from the group comprising of ANA011, AUNP-12, BGB-A317, KD033, pembrolizumab, MCLA-134, mDX400, MEDI0680, muDX400, nivolumab, PDR001, PF-06801591, pidilizumab, REGN-2810, SHR-1210, STI-A1110, TSR-042, ANB011, 244C8, 388D4, TSR042 and XCE853 and the preferred one is pembrolizumab, nivolumab or pidilizumab.

In one embodiment, PD-L1 antagonist is selected from the group comprising of avelumab, BMS-936559, CA-170, durvalumab, MCLA-145, SP142, STI-A1011, STI-A1012, STI-A1010, STI-A1014, A110, KY1003 and atezolimumab and the preferred one is avelumab, durvalumab or atezolimumab.

In one embodiment, PD-L2 antagonist is selected from the group comprising of AMP-224 or rHIgM12B7.

### CTLA4 Antagonists

Suitable anti-CTLA4 antagonist for use in the methods of the invention, include, without limitation, anti-CTLA4 antibodies, human anti-CTLA4 antibodies, mouse anti-CTLA4 antibodies, mammalian anti-CTLA4 antibodies, humanized anti-CTLA4 antibodies, monoclonal anti-CTLA4 antibodies, polyclonal anti-CTLA4 antibodies, chimeric anti-CTLA4 antibodies, MDX-010 (ipilimumab), tremelimumab, anti-CD28 antibodies, anti-CTLA4 adnectins, anti-CTLA4 domain antibodies, single chain anti-CTLA4 fragments, heavy chain anti-CTLA4 fragments, light chain anti-CTLA4 fragments, inhibitors of CTLA4 that agonize the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP 1212422 B. Additional CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17): 10067-10071 (1998); Camacho et al., J. Clin: Oncology, 22(145): Abstract No. 2505 (2004) (antibody CP-675206); Mokyr et al., Cancer Res., 58:5301-5304 (1998), and U.S. Pat. Nos. 5,977,318, 6,682,736, 7,109,003, and 7,132,281.

A preferred clinical CTLA-4 antibody is human monoclonal antibody (also referred to as MDX-010 and ipilimumab with CAS No. 477202-00-9 and available from Medarex, Inc., Bloomsbury, N.J.) is disclosed in WO 01/14424.

With regard to CTLA-4 antagonist (antibodies), these are known and include tremelimumab (CP-675,206) and ipilimumab.

CTLA4 antagonist is selected from group comprising of KAHR-102, AGEN1884, ABR002, KN044, tremelimumab or ipilimumab and the preferred one is tremelimumab or ipilimumab.

### II. Method of Use

The current method of use includes a therapeutic agent that binds and targets ABCB5 (ABCB5 inhibitor, for example, an anti-ABCB5 antibody includes anti-ABCB5 nanobody or small molecule targeting ABCB5) in combination with another therapeutic agent that binds and targets an immune checkpoint molecule (immune checkpoint inhibitor).

The present inventors have discovered for the first time that the co-administration of an ABCB5 inhibitor (for example, anti-ABCB5 antibody) and an immune checkpoint inhibitor (e.g., an antibody or small molecule) effectively inhibits tumor growth synergistically (for example, enhanced T-cell activation or increased IL-2 secretion) in an improved and efficacious manner. In certain embodiments, the combination therapies of the present invention interfere with the metastasis, as well as lack of response to chemotherapy and reduce the side effects associated with the other therapies.

The methods of this invention may find use in treating conditions where enhanced immune stimulation is desired such as by increasing tumor immunogenicity for the treatment of cancer. A variety of cancers may be treated, or their progression may be delayed, which specifically includes solid tumor/cancer.

The present invention discloses a novel immunoinhibitory function of modalities targeted against both ABCB5 and an immune checkpoint molecule (PD-1, PD-L1, PD-L2 or CTLA4) which is important for immune evasion of tumor cells during tumor progression.

In one of the embodiments, the present invention provides a novel approach for treating, preventing or inhibiting the tumors associated with increased level of ABCB5 and/or an immune checkpoint molecule (PD-1, PD-L1, PD-L2 or CTLA4).

The present invention encompasses an enhanced immunoinhibitory or immune modulatory function of a novel combination therapy comprising an ABCB5 inhibitor and immune checkpoint inhibitor which is important for immune evasion of tumor cells during tumor progression.

In one of the embodiment, the present invention provides a novel combination for the treatment of tumors associated with increased levels of ABCB5 and/or an immune checkpoint molecule comprising at least one therapeutic agent that binds and targets both ABCB5 and an immune checkpoint molecule. In a preferred embodiment, a therapeutic agent that binds and targets ABCB5 is an ABCB5 inhibitor which includes an anti-ABCB5 antibody (including an anti-ABCB5 nanobody) or small molecule targeting ABCB5. In another preferred embodiment, a therapeutic agent that binds and targets an immune checkpoint molecule is an immune checkpoint inhibitor.

The word “targets” herein includes the functional inhibition of the said molecule (for example ABCB5) and its depletion or exhaustion within the tumor milieu (for example, ABCB5+ cancer stem cells depletion and inhibition of metastasis or tumorigenesis).

In one embodiment, the present invention embodifies a method of treating or delaying or preventing tumor or cancer in a subject comprising administering to a subject an effective amount of an anti-ABCB5 antibody and an immune checkpoint inhibitor separately, wherein said subject is diagnosed with tumor or cancer associated with increased levels of ABCB5 and/or an immune checkpoint molecule.

Certain methods of the invention relate to methods of targeting ABCB5-mediated efflux of an ABCB5 substrate and/or inducing ADCC (Antibody dependent cellular cytotoxicity) in a cell in a patient in need thereof, by administering an effective amount of an anti-ABCB5 antibody alone or in combination with an immune checkpoint inhibitor.

In some embodiments of the methods, uses, compositions, and kits described herein, the cancer is a solid tumor. In some embodiments, the cancer is urogenital cancers (such as prostate cancer, renal cell cancer, bladder cancer), thyroid cancer, testicular cancer, vulvar cancer, wilm's tumor, rhabdomyosarcoma, retinoblastoma, hormone sensitive or hormone refractory prostate cancer, gynecological cancers (such as ovarian cancer, cervical cancer, endometrial cancer, uterine cancer), lung cancer, non-small cell lung cancer, small cell lung cancer, gastrointestinal stromal cancers, gastrointestinal cancers (such as non-metastatic or metastatic colorectal cancers, pancreatic cancer, gastric cancer, oesophageal cancer, hepatocellular cancer, cholangiocellular cancer), head and neck cancer (such as head and neck squamous cell cancer), malignant glioblastoma, malignant mesothelioma, non-metastatic or metastatic breast cancer (such as hormone refractory metastatic breast cancer, triple negative breast cancer), malignant melanoma, melanoma, metastatic melanoma, merkel cell carcinoma or bone and soft tissue sarcomas, oral squamous cell carcinoma, glioblastoma, brain cancer, osteosarcoma, neuroblastoma, advanced metastatic, an inflammatory myofibroblastic tumor (IMT), cholangiocarcinoma, cystadenocarcionoma, ameloblastoma, chondrosarcoma, dermatofibrosarcoma, ganglioglioma, leiomyosarcoma, medulloblastomma, osteoblastoma and inoperable non-inflammatory locally advanced disease and the like. The most preferred cancer is solid tumor (such as melanoma, metastatic melanoma, oral squamous cell carcinoma, breast cancer, colorectal cancer, glioblastoma, hepatocellular carcinoma) or hematopoietic cancer (leukemia, lymphoma, a lymphocytic leukemia, non-Hodgkin's lymphoma, Hodgkin's lymphoma, an anaplastic large-cell lymphoma, myeloid leukemia, multiple myeloma, acute lymphoblastic leukemia, chronic myeloid leukemia, acute myeloid leukemia).

In some embodiments the methods, uses, compositions and kits described herein, the subject is a human. In some embodiments, the subject has cancer or has been diagnosed with cancer. In some embodiments, the subject is suffering from replaced or refractory cancer (such as solid tumor). In some embodiments, the subject is suffering from solid tumor (such as melanoma, metastatic melanoma, oral squamous cell carcinoma, breast cancer, colorectal cancer, glioblastoma, hepatocellular carcinoma) or hematological cancer (leukemia, lymphoma, a lymphocytic leukemia, non-Hodgkin's lymphoma, Hodgkin's lymphoma, an anaplastic large-cell lymphoma, myeloid leukemia, multiple myeloma, acute lymphoblastic leukemia, chronic myeloid leukemia, acute myeloid leukemia).

In a yet preferred embodiment, the therapeutic combination of the present invention is used to treat or prevent or delay the cancers or tumors such as solid tumor (such as melanoma, metastatic melanoma, oral squamous cell carcinoma, breast cancer, colorectal cancer, glioblastoma, hepatocellular carcinoma) or hematological cancer (leukemia, lymphoma, a lymphocytic leukemia, non-Hodgkin's lymphoma, Hodgkin's lymphoma, an anaplastic large-cell lymphoma, myeloid leukemia, multiple myeloma, acute lymphoblastic leukemia, chronic myeloid leukemia, acute myeloid leukemia).

In some embodiments, the treatment results in a sustained response in the subject after cessation of the treatment. In some embodiments, the subject has cancer that may be at early stage intermediate stage or late stage cancer. In some embodiments, the cancer is metastatic.

The cancers described above can be treated with an ABCB5 inhibitor (for example anti-ABCB5 antibody) and an immune checkpoint inhibitor, which includes the treatment of cancer caused by increased levels of ABCB5 and/or immune checkpoint molecule(s). In some embodiments, the subject treated is suffering from cancer caused by immunosuppression due to the interaction of ABCB5. In some embodiments, the cancer has decreased levels of T-cell infiltration. Increased levels of ABCB5 can be used as a biomarker to select patients undergoing said therapy, therefore ABCB5 would be a marker that would not only guide the expected outcomes of the treatment but also assist in the selection of patients to be appropriately managed at the initial diagnosis to undergo the said therapy.

In one embodiment, the present invention embodies a pharmaceutical composition comprising an effective amount of at least one therapeutic agent that binds and targets both ABCB5 and an immune checkpoint molecule and one or more pharmaceutically acceptable carrier for treating or delaying a tumor/cancer growth or metastasis in a subject.

In another embodiment, the present invention discloses a pharmaceutical composition comprising one or more ABCB5 inhibitor in combination with one or more immune checkpoint inhibitors, along with an optional anti-tumor agent(s) and one or more pharmaceutically acceptable carrier(s) and/or adjuvants.

In one embodiment, the present invention provides a pharmaceutical composition comprising an effective amount of at least one therapeutic agent that binds and targets both ABCB5 and PD1 axis, and one or more pharmaceutically acceptable carrier(s) for treating, preventing or delaying a tumor/cancer growth or metastasis in a subject wherein PD-1 axis includes PD-1, PD-L1, PD-L2.

In another embodiment, the present invention provides a pharmaceutical composition comprising:
(a) an effective amount of an ABCB5 inhibitor(s);
(b) an effective amount of a PD-1 axis antagonist(s),
(c) one or more pharmaceutically acceptable carrier(s) or adjuvant(s),
wherein administering the composition to a subject having a tumor treats, prevents or delays tumor growth or metastasis in the subject.

In yet another embodiment, the present invention provides a pharmaceutical composition comprising an anti-ABCB5 antibody in combination with PD-1 antagonist along with an optional anti-tumor agent(s) and one or more pharmaceutically acceptable carrier(s) or adjuvant(s).

In another embodiment, the present invention provides a pharmaceutical composition comprising an anti-ABCB5 antibody in combination with PD-L1 antagonist along with an optional anti-tumor agent(s) and one or more pharmaceutically acceptable carrier(s) or adjuvant(s).

In another embodiment, the present invention provides a pharmaceutical composition comprising an anti-ABCB5 antibody in combination with PD-L2 antagonist along with an optional anti-tumor agent(s) and one or more pharmaceutically acceptable carrier(s) or adjuvant(s).

In yet another embodiment, the present invention provides a pharmaceutical composition comprising an effective amount of at least one therapeutic agent that binds and targets both ABCB5 and CTLA4 and one or more pharmaceutically acceptable carrier(s) or adjuvant(s) for treating, preventing or delaying a tumor/cancer growth or metastases in a subject.

In another embodiment, the present invention provides a pharmaceutical composition comprising:
(a) an effective amount of an ABCB5 inhibitor(s);
(b) an effective amount of a CTLA4 antagonist(s) and
(c) one or more pharmaceutically acceptable carrier(s) or adjuvant(s)
wherein administering the composition to a subject having a tumor treats, prevents or delays tumor growth or metastasis in the subject.

In another embodiment, the present invention discloses a pharmaceutical composition comprising an anti-ABCB5 antibody in combination with CTLA4 antagonist along with an optional anti-tumor agent(s) and one or more pharmaceutically acceptable carrier(s) and/or adjuvant(s).

In another embodiment, the present invention discloses a method of enhancing, increasing, promoting, modulating desirable immune response in a subject comprising administering to a subject a first composition comprising an effective amount of an ABCB5 inhibitor and a second composition comprising an effective amount of an immune checkpoint inhibitor, wherein said subject is diagnosed with tumor or cancer associated with increased levels of ABCB5 and/or immune checkpoint molecule(s).

In some embodiments, provided is a method for treating, preventing or delaying progression of cancer in a subject comprising administering to the subject an effective amount of an ABCB5 inhibitor and an immune checkpoint inhibitor, further comprising administering an additional therapy. The additional therapy may be radiation therapy, surgery (such as lumpectomy and a mastectomy), chemotherapy, gene therapy, DNA therapy, viral therapy, RNA therapy, immunotherapy, bone marrow transplantation, nanotherapy, or a combination of the foregoing. The additional therapy may be in the form of adjuvant or neoadjuvant therapy. In some embodiments, the additional therapy is the administration of small molecule enzymatic inhibitor or anti-metastatic agent. In some embodiments, the additional therapy is the administration of side-effect limiting agents (such as agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. The additional therapy may be one or more of the anti-tumor agents described herein below.

The anti-tumor agent may be selected from the group consisting of an antibody, an antimicrotubule agents, topoisomerase inhibitors, anti-metabolites, mitotic inhibitors, alkylating agents, anthracyclines, vinca alkaloids, a taxane, an anthracycline, a platin derivative, a small molecule, a kinase inhibitor, an alkylating agent, a mTOR inhibitor, intercalating agents, agents capable of interfering with a signal transduction pathway, agents that promote apoptosis, proteosome inhibitors, and radiation (e.g., local or whole body irradiation (e.g., gamma irradiation). Examples of anti-tumor agents include but not limited: docetaxel, paclitaxel, doxorubicin, farmorubicin, cyclophosphamide, 5-fluorouracil, vinorelbine, cisplatin, carboplatin, trastuzumab, bevacizumab, cetuximab, panitumumab, sunitinib, sorafenib, gefitinib, erlotinib, temsirolimus, adotrastuzumab, emtansine, crizotinib, pertuzumab, ramucirumab, regorafenib, vemurafenib, abiraterone acetate, ziv-aflibercept and the like. Alternatively, or in combination with the aforesaid combinations, the methods and compositions described herein can be administered in combination with one or more of: a vaccine, e.g., a therapeutic cancer vaccine; or other forms of cellular immunotherapy.

In another embodiment, provided herein is use of an ABCB5 inhibitor (for example anti-ABCB5 antibody) in the manufacture of a first pharmaceutical composition for treating, preventing or delaying progression of tumor in a subject, wherein the first pharmaceutical composition comprises the ABCB5 inhibitor (for example anti-ABCB5 antibody) and one or more pharmaceutically acceptable carrier(s), and wherein the treatment comprises administration of the first pharmaceutical composition in combination with a second pharmaceutical composition comprising an immune checkpoint inhibitor and one or more pharmaceutically acceptable carrier(s).

In another embodiment, provided herein is a first pharmaceutical composition comprising an ABCB5 inhibitor (for example anti-ABCB5 antibody) and one or more pharmaceutically acceptable carrier(s) for use in treating or delaying progression of tumor in a subject, wherein the treatment comprises administration of said first pharmaceutical composition in combination with a second composition, wherein the second composition comprises an immune checkpoint inhibitor and one or more pharmaceutically acceptable carrier(s).

In another embodiment, provided herein is a second pharmaceutical composition comprising an immune checkpoint inhibitor and one or more pharmaceutically acceptable carrier(s) for use in treating or delaying progression of tumor in a subject, wherein the treatment comprises administration of said second pharmaceutical composition in combination with a first composition, wherein the first composition comprises an ABCB5 inhibitor (for example anti-ABCB5 antibody) and one or more pharmaceutically acceptable carrier(s).

In another embodiment, provided herein is use of an ABCB5 inhibitor (for example anti-ABCB5 antibody) in the manufacture of a first pharmaceutical composition for enhancing immune function in a subject having cancer or tumor, wherein the first pharmaceutical composition comprises the ABCB5 inhibitor (for example anti-ABCB5 antibody) and one or more pharmaceutically acceptable carrier(s), and wherein treatment comprises administration of the pharmaceutical composition in combination with a second composition comprising an immune checkpoint inhibitor and one or more pharmaceutically acceptable carrier(s).

In another embodiment, provided herein is use of an immune checkpoint inhibitor in the manufacture of a second pharmaceutical composition for enhancing immune function in a subject having cancer, wherein the second pharmaceutical composition comprises the immune checkpoint inhibitor and one or more pharmaceutically acceptable carrier(s), and wherein the treatment comprises administration of the second pharmaceutical composition in combination with a first composition comprising an ABCB5 inhibitor (for example anti-ABCB5 antibody) and one or more pharmaceutically acceptable carrier(s).

In another embodiment, the present invention provides a combination therapy for the treatment of tumor or cancer, the said combination comprises(a) an ABCB5 inhibitor selected from anti-ABCB5 antibody (including anti-ABCB5 nanobody) or small molecule targeting ABCB5 and (b) an immune checkpoint inhibitor selected from the group comprising of PD-1 antagonist, PD-L1 antagonist, PD-L2 antagonist or CTLA4 antagonist.

In some embodiments, the present invention provides a method for identifying a patient diagnosed with increased levels of ABCB5 and an immune checkpoint molecule(s) having an increased probability of obtaining improved overall survival following co-administration treatment therapy with an ABCB5 inhibitor and an immune checkpoint inhibitor(s).

In another embodiment, the present invention provides methods of preventing and/or treating a tumor associated with increased levels of ABCB5 and/or immune checkpoint molecule, said method comprising: (a) administering to a subject in need thereof one or more doses of an effective amount of one or more therapeutic agents of the invention; and (b) monitoring the plasma level/concentration of the said administered therapeutic agents in said subject after administration of a certain number of doses of the said therapeutic agents. Moreover, preferably, said certain number of doses is 1, 2, 3, 4, 5, 6, 7, or 8 doses of an effective amount of therapeutic agents of the invention,

In some embodiments, the present invention relates to methods of detecting an immune checkpoint molecule (PD-1, PD-L1, PD-L2 or CTLA4) or ABCB5 expressing cells by contacting cells with the immune checkpoint inhibitor or the ABCB5 inhibitor (for example, anti-ABCB5 antibody) under conditions to permit the formation of a complex between the cell and the antibody.

The present invention provides, for example, isolated antibodies, methods of making such antibodies or small molecules. It also provides methods of making such pharmaceutical compositions containing the antibodies or small molecules of the present invention.

The present invention comprises anti-ABCB5 antibodies (and fragments thereof) that compete with antibodies having at least one region that binds to epitope corresponding to amino acid residues 1-30 of human ABCB5 for binding to ABCB5. The present invention provides an anti-ABCB5 antibody or antigen-binding fragment thereof, having at least one region that binds to epitope corresponding to amino acid residues 481-674 of the human ABCB5, wherein the anti-ABCB5 antibody or antigen binding fragment competitively inhibits the binding of antibodies having at least one region that binds to epitope corresponding to amino acid residues 1-30 of the human ABCB5 to ABCB5.

### III. Administration

Suitable administration/treatment protocols for treating cancer or tumor in a subject include, for example, administering to the patient an effective amount of an ABCB5 inhibitor (for example, anti-ABCB5 antibody) and an immune checkpoint inhibitor.

In some embodiments, the combination therapy of the invention comprises administration of an ABCB5 inhibitor (for example anti-ABCB5 antibody) and an immune checkpoint inhibitor. The ABCB5 inhibitor and the immune checkpoint inhibitor may be administered in any suitable manner known in the art. For example, the ABCB5 inhibitor and the immune checkpoint inhibitor may be administered sequentially (at different times) or concurrently (at the same time).

In some embodiments, the immune checkpoint inhibitor is administered before administration of the ABCB5 inhibitor (for example anti-ABCB5 antibody). In some embodiments, the immune checkpoint inhibitor is administered simultaneously with administration of the ABCB5 inhibitor. In some embodiments, the immune checkpoint inhibitor is administered after administration of the ABCB5 inhibitor.

In some embodiments, the ABCB5 inhibitor or an immune checkpoint inhibitor is administered continuously. In some embodiments, the ABCB5 inhibitor or immune checkpoint inhibitor is administered intermittently.

In some embodiments, the immune checkpoint inhibitor and the ABCB5 inhibitor is co-administered, for example, the administration of said immune checkpoint inhibitor and the ABCB5 inhibitor (for example anti-ABCB5 antibody) as two separate formulations. The co-administration can be simultaneous or sequential in either order. In one further embodiment, there is a time period while both (or all) antibodies simultaneously exert their biological activities. Said immune checkpoint inhibitor and ABCB5 inhibitor (for example anti-ABCB5 antibody) are co-administered either simultaneously or sequentially for example, intravenous (i.v.) through a continuous infusion. When both therapeutic agents are co-administered sequentially the therapeutic agents are administered in two separate administrations that are separated by a “specific period of time”. The term specific period of time is meant anywhere from 1 hour to 30 days. For example, one of the agents can be administered within about 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 day, or 24, 23,22, 21, 20, 19, 18, 17, 16, 15, 14, 13, 12, 11, 10, 9, 8, 7, 6, 5, 4, 3, 2 or 1 hour from the administration of the other therapeutic agent, and, in one embodiment, the specific period time is 10, 9, 8, 7, 6, 5, 4, 3, 2, or 1 day, or 24, 23, 22, 21, 20, 19, 18, 17, 16, 15, 14, 13, 12, 11, 10, 9, 8, 7, 6, 5,4, 3, 2 or 1 hour. In some embodiments, simultaneous administration means at the same time or within a short period of time, usually less than 1 hour.

A dosing period as used herein is meant for a period of time, during which each antibody has been administered at least once. A dosing period is usually about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, and, in one embodiment, 6, 7, 8, 9, 10, 11, 12, 13, or 14 days, for example, 7 or 14 days.

In certain embodiments, multiple (for example, 2, 3, 4, 5, 6, 7, 8, 9, 10 or more) doses of a ABCB5 inhibitor (for example an anti-ABCB5 antibody) and multiple (for example, 2, 3, 4, 5, 6, 7, 8, 9, 10 or more) doses of an immune checkpoint inhibitor are administered to a subject in need of treatment.

In certain embodiments, the immune checkpoint inhibitor is administered in a dose of 0.01 mg/kg, 0.05 mg/kg, 0.1 mg/kg, 0.2 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg or 30 mg/kg. The dose of the immune checkpoint inhibitor may vary from about 0.01 mg/kg to 30 mg/kg, preferably 0.1 mg/kg to 20 mg/kg, more preferably 1 mg/kg to 10 mg/kg. In certain embodiments, the immune checkpoint inhibitor is administered by injection (e.g., subcutaneously or intravenously) at a dose of about 0.01 mg/kg to 30 mg/kg, e.g., about 0.1 mg/kg to 20 mg/kg, about 1 mg/kg to 10 mg/kg, about 1 mg/kg to 5 mg/kg., or about 1 to 3 mg/kg.

In certain embodiments, the checkpoint inhibitor is administered one dose per day, one dose every 2 days, one dose every 3 days, one dose every 4 days, one dose every 5 days, once a week, once every two weeks, once every three weeks or once every four weeks, preferably one dose every 3 days. In certain embodiments, the checkpoint inhibitor is administered as a single dose, in two doses, in three doses, in four doses, in five doses, or in 6 or more doses. The dosing schedule can vary from e.g., once a week to once every 2, 3, or 4 weeks. In one embodiment, the immune checkpoint inhibitor is administered at a dose from about 1 mg/kg to 10 mg/kg every other week.

In certain embodiments, the ABCB5 inhibitor (for example anti-ABCB5 antibody) is administered in a dose of 0.1 mg/kg, 0.2 mg/kg, 0.3 mg/kg, 0.5 mg/kg, 0.7 mg/kg, 0.8 mg/kg, 1 mg/kg, 2 mg/kg, 2.1 mg/kg, 3 mg/kg, 4 mg/kg, 5 mg/kg, 6 mg/kg, 7 mg/kg, 8 mg/kg, 9 mg/kg, 10 mg/kg, 15 mg/kg or 20 mg/kg. In another embodiment, the dosage of an ABCB5 inhibitor of the invention administered to prevent and/or treat a cancer associated with increased levels of ABCB5 in a patient is a unit dose of about 0.1 mg/kg to about 20 mg/kg, about 0.1 mg/kg to about 10 mg/kg, about 0.1 mg/kg to about 8 mg/kg, about 0.1 mg/kg to about 7 mg/kg, about 0.1 mg/kg to about 6 mg/kg, about 0.1 mg/kg to about 5 mg/kg, about 0.1 mg/kg to about 4 mg/kg, preferably, about 0.1 mg/kg to about 3 mg/kg, about 0.2 mg/kg to 3 mg/kg, about 0.3 mg/kg to about 3 mg/kg, about 0.4 mg/kg to about 3 mg/kg, about 0.6 mg/kg to about 3 mg/kg, about 0.8 mg/kg to about 3 mg/kg, about 0.1 mg/kg to 2 mg/kg, about 0.1 mg/kg to 1 mg/kg. Total daily dose may vary from 20 mg to 200 mg, preferably 50 mg to 150 mg, most preferably 80 mg to 140 mg. The dose of an anti-ABCB5 antibody may vary from about 0.1 mg/kg to 20 mg/kg, preferably 0.50 mg/kg to 10 mg/kg, more preferably 1 mg/kg to 5 mg/kg. In a preferred embodiment, an anti-ABCB5 antibody of the present invention is administered at a unit dose of about 0.1 mg/kg, about 0.2 mg/kg, about 0.4 mg/kg, about 0.6 mg/kg, about 0.8 mg/kg, about 1 mg/kg, about 2 mg/kg, about 3 mg/kg, about 4 mg/kg or 5 mg/kg. In one embodiment, the ABCB5 inhibitor is administered at a dose from about 1 mg/kg to 10 mg/kg biweekly.

In certain embodiments, the ABCB5 inhibitor is administered one dose per day, one dose every 2 days, one dose every 3 days, one dose every 4 days, one dose every 5 days, once a week, once every two weeks, or once every four weeks, preferably one dose every 3 days. In certain embodiments, the ABCB5 inhibitor is administered as a single dose, in two doses, in three doses, in four doses, in five doses, or in 6 or more doses. The dosing schedule can vary from e.g., once a week to once every 2, 3, or 4 weeks. In one embodiment, the ABCB5 inhibitor is administered at a dose from about 0.50 mg/kg to 10 mg/kg every other week. In certain embodiments the dose frequency may vary from once a day to once very month.

An effective amount of the ABCB5 inhibitor (for example, anti-ABCB5 antibody) and the immune checkpoint inhibitor may be administered for prevention or treatment of cancer. The appropriate dosage of the ABCB5 inhibitor (for example, anti-ABCB5 antibody) and/or the immune checkpoint inhibitor may be determined based on the type of disease to be treated, the type of the ABCB5 inhibitor and the immune checkpoint inhibitor, the severity and course of the disease, the clinical condition of the subject, the subject's clinical history and response to the treatment, the symptoms involved, the subject's body mass, gender, immune status and the discretion of the attending physician. Suitable regimens can be selected by one skilled in the art by considering such factors and by following, for example, dosages reported in literature and recommended in the Physician's Desk Reference (59th ed., 2005).

Preferably, the dosages of therapeutic agents used in combination therapies of the invention are lower than those which have been or are currently being used to prevent and/or treat a tumor associated with increased levels of ABCB5 and/or immune checkpoint molecule.

In some embodiments, a method of treating cancer will be performed even with a low likelihood of success, but which, given the medical history and estimated survival expectancy of a patient, is nevertheless deemed to induce an overall beneficial course of action.

Accordingly, in one embodiment, the dose of the ABCB5 inhibitor and immune checkpoint inhibitor is calculated as mg/kg body weight. However, in another embodiment, the dose of the ABCB5 inhibitor and/or immune checkpoint inhibitor is a flat fixed dose that is fixed irrespective of the weight of the patient.

The ABCB5 inhibitor (for example, anti-ABCB5 antibody) and the immune checkpoint inhibitor may be administered by the same route of administration or by different routes of administration. In some embodiments, the ABCB5 inhibitor is administered intravenously, intramuscularly, subcutaneously, topically, orally, transdermally, intraperitoneally, intraorbitally, by implantation, by inhalation, intrathecally, intraventricularly, or intranasally. In some embodiments, the immune checkpoint inhibitor is administered intravenously, intramuscularly, subcutaneously, topically, orally, transdermally, intraperitoneally, intraorbitally, by implantation, by inhalation, intrathecally, intraventricularly, or intranasally.

In some embodiments, the immune checkpoint inhibitor is a PD-L1 antagonist (for example anti-PD-L1 antibody). In some embodiments, the anti-PD-L1 antibody is administered to the subject intravenously at a dose of 120 mg once every three weeks. In some embodiments, the anti-PD-L1 antibody is administered with an ABCB5 inhibitor (for example, anti-ABCB5 antibody).

### IV. Pharmaceutical Composition/Formulations

Also provided herein are pharmaceutical compositions or formulations comprising an ABCB5 inhibitor (for example, anti-ABCB5 antibody) and/or an immune checkpoint inhibitor and one or more pharmaceutically acceptable carrier(s) or adjuvant(s). The anti-ABCB5 antibody and the immune checkpoint inhibitor can be present in a single composition or as two or more different compositions and can be administered via the same administration route or via different administration routes. In one embodiment, the pharmaceutical combination comprises the anti-ABCB5 antibody and the immune checkpoint inhibitor separately or together.

In one embodiment, the present invention provides a composition comprising an ABCB5 inhibitor (for example, anti-ABCB5 antibody) and one or more pharmaceutically acceptable carrier(s). Any of the pharmaceutically acceptable carrier described herein or known in the art may be used.

In a still further embodiment, the invention provides for a composition comprising an immune checkpoint inhibitor such as a PD-1 antagonist, PD-L1 antagonist, or a PD-L2 antagonist or a CTLA4 antagonist as provided herein and one or more pharmaceutically acceptable carrier(s) or adjuvant(s). Any of the pharmaceutically acceptable carrier described herein or known in the art maybe used.

As used herein, the term “pharmaceutical composition” refers to a composition comprising at least one active therapeutic agent (for example, an ABCB5 inhibitor or an immune checkpoint inhibitor) and one or more pharmaceutically acceptable carrier(s). Pharmaceutically acceptable carriers or adjuvants are well known to the skilled in the art, and usually depend on the chosen route of administration, even water is included as an example of carrier or adjuvant. In some embodiments, the mixture comprises at least one ABCB5 inhibitor (for example, anti-ABCB5 antibody) in an amount that results in an additive or a synergistic effect with at least one immune checkpoint inhibitor in a subject when both are administered simultaneously (for example, in a single formulation or concurrently as separate formulations). In some embodiments, a first composition comprising an ABCB5 inhibitor (for example, anti-ABCB5 antibody) and one or more pharmaceutically acceptable carrier(s) and a second composition comprising an immune checkpoint inhibitor and one or more pharmaceutically acceptable carrier(s) wherein both are present in an amount that results in an additive or a synergistic effect when both are administered sequentially (as a separate formulations) to the subject. In another preferred embodiment, the present combination used for treating, prevention and ameliorating the tumor is administered subcutaneously or intravenously.

Pharmaceutical compositions suitable for administration to human patients are typically formulated for parenteral administration, e.g., in a liquid carrier, or suitable for reconstitution into liquid solution or suspension for parenteral administration. In general, such compositions typically comprise a pharmaceutically acceptable carrier. As used herein, the term “pharmaceutically acceptable” means approved by a government regulatory agency or listed in the U.S. Pharmacopeia or another generally recognized pharmacopeia for use in animals, particularly in humans. Pharmaceutical compositions and formulations as described herein can be prepared by mixing the therapeutic agent (for example, antibody) having the desired degree of purity with one or more pharmaceutically acceptable carrier(s) (Remington's Pharmaceutical Sciences 16^{th} edition, Osol, A. Ed. (1980)), in the form of lyophilized formulations or aqueous solutions. The term “carrier” refers to a diluent, adjuvant, excipient, or vehicle with which the compound is administered. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride, hexamethonium chloride, benzalkonium chloride, benzethonium chloride, phenol, butyl or benzyl alcohol; chlorobutanol; thimerosal's, alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; chelating agents such as EDTA; monosaccharides, disaccharides, and other carbohydrates including sugars such as sucrose, mannitol, trehalose or sorbitol, glucose, mannose, or dextrins; salt-forming counter-ions such as sodium; metal complexes (for example., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20hyaluronidase glycoproteins, such as rHuPH20 (HYLENEX®, Baxter International, Inc.). The carrier can be a solvent or reconstitution medium or dispersion medium containing, for example, water, ethanol, polyol (for example, glycerol, propylene glycol, and liquid polyethylene glycol, and the like), and suitable mixtures thereof. For intravenous administration, suitable carriers include physiological saline, bacteriostatic water, Cremophor EL (BASF, Parsippany, N.J.) or phosphate buffered saline (PBS). Prolonged absorption of the injectable compositions can be brought about by including in the composition an agent which delays absorption, for example, aluminum monostearate and gelatin.

Solutions or suspensions used for subcutaneous application typically include one or more of the following components: a sterile carrier such as water for injection, saline solution, fixed oils, polyethylene glycols, glycerin, propylene glycol, or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose. The pH can be adjusted with acids or bases, such as hydrochloric acid or sodium hydroxide. Such preparations may be enclosed in ampoules, disposable syringes or multiple dose vials made of glass or plastic. The present invention also provides other formulations such as microcapsules, nanoparticles or sustained release compositions, intranasal compositions, oral compositions. Active agents may be entrapped in microcapsules prepared, for example, by coacervation techniques or by interfacial polymerization, for example, hydroxymethylcellulose or gelatin microcapsules and poly-(methylmethacylate) microcapsules, respectively, in colloidal drug delivery systems (for example, liposomes, albumin microspheres, microemulsions, nanoparticles and nano-capsules) or in macro emulsions. Such techniques are disclosed in Remington's Pharmaceutical Sciences 16^{th} edition, Osol, A. Ed. (1980). In certain embodiments, the presently disclosed therapeutic agents are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions containing the presently disclosed antibodies can also be used as pharmaceutically acceptable carriers. Suitable examples of sustained release preparations include semipermeable matrices of solid hydrophobic polymers containing the therapeutic agent (for example, antibody) wherein the matrices are in the form of shaped articles, e.g. films, or microcapsules. The formulations to be used for in vivo administration are generally sterile. Sterility may be readily accomplished, e.g., by filtration through sterile filtration membranes.

For oral use, the pharmaceutical compositions of the present invention, may be administered, for example, in the form of tablets or capsules, powders, dispersible granules, or cachets, or as aqueous solutions or suspensions. Oral compositions generally include an inert carrier (for example, diluent) or an edible carrier. They can be enclosed in gelatin capsules or compressed into tablets. For oral administration, the antibodies can be combined with carriers and used in the form of tablets, troches, or capsules. Pharmaceutically compatible binding agents, and/or adjuvant materials can be included as part of the composition. The tablets, pills, capsules, troches, and the like can contain any of the following ingredients, or compounds of a similar nature; a binder such as microcrystalline cellulose, gum tragacanth or gelatin; an excipient such as starch or lactose, a disintegrating agent such as alginic acid, primogel, or corn starch; a lubricant such as magnesium stearate or stearates; a glidant such as colloidal silicon dioxide; a sweetening agent such as sucrose or saccharin; or a flavoring agent such as peppermint, methyl salicylate, or orange flavoring.

Liquid preparations may also include solutions for intranasal administration. Aerosol preparations suitable for inhalation may include solutions and solids in powder form, which may be in combination with a pharmaceutically acceptable carrier, such as an inert compressed gas.

The amount of ABCB5 inhibitor (for example, anti-ABCB5 antibody) present in a composition should, in general, be in the range of about 0.01 to about 30% w/w and preferably in an amount of 0.5 to 20% w/w of the composition. Similarly, the amount of an immune checkpoint inhibitor present in a composition in the range of about 0.01 to about 30% w/w and preferably in an amount of 0.5 to 20% w/w of the composition. The immune checkpoint inhibitor is selected from the group comprising of PD-1 antagonist, PD-L1 antagonist, PD-L2 antagonist, CTLA4 antagonist. The precise dose to be employed in the formulation will also depend on the route of administration, and the seriousness of the cancer, and should be decided according to the judgment of the practitioner and each patient's circumstances. Effective doses may be extrapolated from dose-response curves derived from in vitro or animal model test systems. In some embodiments, the PD-L1 antagonist (for example, anti-PD-L1 antibody) described herein is in formulation comprising the antibody at an amount of about 60 mg/mL, histidine acetate in a concentration of about 20 mM, sucrose in a concentration of about 120 mM, and polysorbate (e.g., polysorbate 20) in a concentration of 0.04% (w/v), and the formulation has a pH of about 5.8. In some embodiments, the anti-PD-L1 antibody described herein is in a formulation comprising the antibody in an amount of about 125 mg/mL, histidine acetate in a concentration of about 20 mM, sucrose is in a concentration of about 240 mM, and polysorbate (e.g. polysorbate 20) in a concentration of 0.02% (w/v), and the formulation has a pH of about 5.5.

In some embodiments, the ABCB5 inhibitor (for example, anti-ABCB5 antibody) described herein is in formulation comprising an effective amount of an ABCB5 inhibitor (for example, anti-ABCB5 antibody), and one or more pharmaceutically acceptable carrier(s) or adjuvant(s) selected from the group comprising bulking agent, buffer, surfactant, pH modifier and the formulation has an appropriate pH.

In certain embodiments, the various processes of making above mentioned formulations or compositions are included and such compositions can be manufactured by any of the processes known in the art.

### V. Kits

In some embodiments, a combination includes a formulation of an ABCB5 inhibitor and the immune checkpoint inhibitor, with or without instructions for combined use or to combination products. The combined therapeutics can be manufactured and/or formulated by the same or different manufacturers. The combination therapeutics may thus be entirely separate pharmaceutical dosage forms or pharmaceutical compositions that are also sold independently of each other. In embodiments, instructions for their combined use are provided: (i) prior to release to physicians (e.g. in the case of a “kit of part” comprising a first therapeutic agent and the other therapeutic agent), (ii) by the physicians themselves (or under the guidance of a physician) shortly before administration; (iii) the patient themselves by a physician or medical staff.

In another aspect, provided is a kit comprising an ABCB5 inhibitor (for example, anti-ABCB5 antibody) and/or an immune checkpoint inhibitor for treating or delaying progression of a cancer in subject or for enhancing immune function of a subject having cancer. In some embodiments, the kit comprises an ABCB5 inhibitor and a package insert comprising instructions for using the ABCB5 inhibitor in combination with an immune checkpoint inhibitor to treat or delay progression of cancer in a subject or to enhance immune function of a subject having cancer. In some embodiments, the kit comprises an immune checkpoint inhibitor and a package insert comprising instructions for using the immune checkpoint inhibitor in combination with an ABCB5 inhibitor to treat or delay progression of cancer in a subject or to enhance immune function of a subject having cancer. In some embodiments, the kit comprises an ABCB5 inhibitor and an immune checkpoint inhibitor, and a package insert comprising instructions for using the ABCB5 inhibitor and the immune checkpoint inhibitor to treat or delay progression of cancer in a subject or to enhance immune function of a subject having cancer. Any of the ABCB5 inhibitor (for example, anti-ABCB5 antibody) and/or immune checkpoint inhibitors described herein may be included in the kits.

In some embodiments, the kit comprises a container containing one or more of the ABCB5 inhibitor (for example, anti-ABCB5 antibody) and immune checkpoint inhibitors described herein. Suitable containers include, for example, bottles, vials (e.g., dual chamber vials), syringes (such as single or dual chamber syringes) and test tubes. The container may be formed from a variety of materials such as glass or plastic. In some embodiments, the kit may comprise a label (e.g., on or associated with the container) or a package insert. The label or the package insert may indicate that the compound contained therein may be useful or intended for treating or delaying progression of cancer in a subject or for enhancing immune function of a subject having cancer. The kit may further comprise other materials desirable from a commercial and user standpoint, including other buffers, diluents, filters, needles, and syringes. In one embodiment of the invention, an immune checkpoint inhibitor is PD-1 antagonist, PD-L1 antagonist, PD-L2 antagonist or CTLA4 antagonist.

Thus, in some embodiments, the present invention is directed to kits which comprise a first composition comprising the one or more ABCB5 inhibitors (for example, anti-ABCB5 antibody), and a second composition comprising one or more immune checkpoint inhibitors. In some embodiments, the first and second composition may be mixed together before administering to the subject. In some embodiments, the first and second compositions, may be administered either simultaneously or sequentially (i.e., spaced out over a period of time) so as to obtain the maximum efficacy, additivity, synergy, or a combination thereof of the combination.).

The dosage regimen of the active principles and of the pharmaceutical composition described herein can be chosen by prescribing physicians, based on their knowledge of the art, including information published by regulatory authorities. For example, Nivolumab (Opdivo®) is typically administered intravenously. According to the U.S. Food and Drug Administration (FDA), the recommended dose of Opdivo® is 3 mg/kg administered as an intravenous infusion over 60 minutes every 2 weeks until disease progression.

In some embodiments of the methods, uses, compositions, and kits described herein, the immune checkpoint inhibitor is selected from the group consisting of a PD-1 antagonist, a PD-L1 antagonist and a PD-L2 antagonist. In some embodiments, the PD-laxis binding antagonist is a PD-1 antagonist. In some embodiments, the anti PD-1 antagonist inhibits the binding of PD-1 to its ligand binding partners. In some embodiments, the PD-1 antagonist inhibits the binding of PD-1 to PD-L1, PD-1 to PD-L2, or PD-1 to both PD-L1 and PD-L2.

### VI. Outcomes

In one embodiment, the treatment produces at least one therapeutic effect selected from the group consisting of reduction in size of a tumor, reduction in a number of metastatic lesions over time, complete response, partial response and stable disease. In yet another embodiment, one or more of the following can occur: the number of cancer cells can be reduced, tumor size can be reduced, cancer cell infiltration into peripheral organs can be inhibited, retarded, slowed or stopped; tumor metastases can be inhibited or slowed, tumor growth can be inhibited, T-cell activation as read out by Interleukin-2 secretion can be increased.

In another embodiment, administration of an ABCB5 inhibitor (for example anti-ABCB5 antibody) and an immune checkpoint inhibitor results in at least a three-fold reduction (e.g., a 3.5-fold reduction) in tumor volume, e.g., relative to treatment with the ABCB5 inhibitor or the immune checkpoint inhibitor alone or relative to tumor growth on the first day of treatment or immediately before initiation of treatment.

In a further embodiment, administration of an ABCB5 inhibitor (for example anti-ABCB5 antibody) and an immune checkpoint inhibitor results in tumor growth inhibition of at least 80%, e.g., relative to treatment with the ABCB5 inhibitor (for example anti-ABCB5 antibody) or an immune checkpoint inhibitor alone or relative to tumor growth on the first day of treatment or immediately before initiation of treatment.

In certain embodiments, administration of an ABCB5 inhibitor (for example anti-ABCB5 antibody) and an immune checkpoint inhibitor reduces tumor mass by at least 10%, 20%, 30%, 40%, 50%, 60%, 70%, 80%, 90%, 95%, 99% relative to the tumor mass prior to initiation of the treatment or on the first day of treatment. In some embodiment, the tumor mass is no longer detectable following treatment as described herein. In some embodiments, a subject is in partial or full remission.

In one embodiment, the combination therapy of the present invention is being tested in the mouse model of the relevant cancer. Those of skill in the art should, in light of the present disclosure, appreciate that many changes or variations can be made in the specific embodiments which are disclosed and still obtain a like or similar result without departing from the spirit and scope of the invention. The present invention is not to be limited in scope by the specific embodiments described herein (which are intended only as illustrations of aspects of the invention), and functionally equivalent methods and components are within the scope of the invention. Indeed, various modifications of the invention, in addition to those shown and describe herein, will become apparent to those skilled in the art from the foregoing description.

The following examples are provided to further illustrate the embodiments of the present invention, but are not intended to limit the scope of the invention. While they are typical of those that might be used, other procedures, methodologies, or techniques known to those skilled in the art may alternatively be used.

The following embodiments further describe the objects of the present invention in accordance with the best mode of practice, however, disclosed invention is not restricted to the particular embodiments hereinafter described.

### Specific Embodiments of the Present Invention:

Embodiment 1: A method of enhancing an immune response in a subject, comprising administering to the subject an effective amount of at least one therapeutic agent that binds and targets both ABCB5 and an immune checkpoint molecule(s).

Embodiment 2. A method of enhancing an immune response in a subject, comprising administering to the subject an effective amount of a therapeutic agent that binds and targets ABCB5 in combination with a second therapeutic agent that binds and targets an immune checkpoint molecule(s).

Embodiment 3: The method according to embodiments 1 and 2, wherein therapeutic agent is selected from a group comprises of an antibody including nanobody, or small molecule and the preferred one is monoclonal antibody or nanobody.

Embodiment 4. The method according to embodiments 1 and 2, wherein the subject has been diagnosed as having tumor associated with increased levels of ABCB5 and/or an immune checkpoint molecule(s).

Embodiment 5. The method according to embodiments 1, 2 and 4, wherein said immune checkpoint molecule is selected from the group comprising of CTLA4 and PD1 axis which further includes PD-1, PD-L1, PD-L2.

Embodiment 6. The method according to embodiments 1 to 3, wherein the therapeutic agent that binds and targets ABCB5 is an ABCB5 inhibitor selected from the group comprising of an anti-ABCB5 antibody including an anti-ABCB5 nanobody, or small molecule targeting ABCB5 and combination thereof.

Embodiment 7. The method according to embodiments 1 to 3, wherein the therapeutic agent that binds and targets an immune checkpoint molecule is an immune checkpoint inhibitor selected from the group comprising of PD1 antagonist, PD-L1 antagonist, PD-L2 antagonist, CTLA4 antagonist and combination thereof.

Embodiment 8. The method according to embodiment 6, wherein anti-ABCB5 antibody is a monoclonal antibody that binds and targets the amino acid residues corresponding to 481-674 of the human ABCB5.

Embodiment 9. The method according to embodiment 7, wherein the PD-1 antagonist is selected from the group comprising of ANA011, AUNP-12, BGB-A317, KD033, pembrolizumab, MCLA-134, mDX400, MEDI0680, muDX400, nivolumab, PDR001, PF-06801591, pidilizumab, REGN-2810, SHR-1210, STI-A1110, TSR-042, ANB011, 244C8, 388D4, TSR042 or XCE853 and the preferred one is pembrolizumab, nivolumab or pidilizumab.

Embodiment 10. The method according to embodiment 7, wherein the PD-L1 antagonist is selected from the group comprising of avelumab, BMS-936559, CA-170, durvalumab, MCLA-145, SP142, STI-A1011, STI-A1012, STI-A1010, STI-A1014, A110, KY1003 or atezolimumab and the preferred one is avelumab, durvalumab or atezolimumab.

Embodiment 11. The method according to embodiment 7, wherein the PD-L2 antagonist is selected from the group comprising of AMP-224 or rHIgM12B7.

Embodiment 12. The method according to embodiment 7, wherein CTLA4 antagonist is selected from the group comprising of KAHR-102, AGEN1884, ABR002, KN044, tremelimumab or ipilimumab and the preferred one is tremelimumab or ipilimumab.

Embodiment 13. The method according to embodiments 1, 2 and 4, wherein the subject has tumor selected from the group comprising of melanoma, metastatic melanoma, oral squamous cell carcinoma, breast cancer, colorectal cancer, glioblastoma, hepatocellular carcinoma, leukemia, lymphoma, a lymphocytic leukemia, non-Hodgkin's lymphoma, Hodgkin's lymphoma, an anaplastic large-cell lymphoma, myeloid leukemia, multiple myeloma, acute lymphoblastic leukemia, chronic myeloid leukemia or acute myeloid leukemia.

Embodiment 14. The method according to embodiment 13, wherein the subject has metastatic tumor.

Embodiment 15. A pharmaceutical composition comprising:
(a) an effective amount of an ABCB5 inhibitor(s);
(b) an effective amount of an immune checkpoint inhibitor(s) and
(c) one or more pharmaceutically acceptable carrier(s) or adjuvants(s)
wherein administering the composition to a subject having a tumor treats, prevents or delays tumor growth or metastasis in the subject.

Embodiment 16. A pharmaceutical composition comprising:
(a) an effective amount of an ABCB5 inhibitor(s);
(b) an effective amount of an immune checkpoint inhibitor(s);
(c) an optional anti-tumor agent(s) and
(d) one or more pharmaceutically acceptable carrier(s) or adjuvant(s)
wherein administering the composition to a subject having a tumor treats, prevents or delays tumor growth or metastasis in the subject.

Embodiment 17. A pharmaceutical composition for use in combination with an immune checkpoint inhibitor comprising of PD-1 antagonist, PD-L1 antagonist, PD-L2 antagonist and CTLA4 antagonist for treating a tumor, wherein said pharmaceutical composition comprises an ABCB5 inhibitor with one or more pharmaceutically acceptable carrier(s) or adjuvant(s).

Embodiment 18. The pharmaceutical composition according to embodiments 15 to 17, wherein ABCB5 inhibitor includes an anti-ABCB5 antibody including an anti-ABCB5 nanobody, or small molecule targeting ABCB5 and the preferred one is an anti-ABCB5 antibody including anti-ABCB5 antibody which is a monoclonal that binds and targets the amino acid residues corresponding to 481-674 of the human ABCB5.

Embodiment 19. The pharmaceutical composition according to embodiments 15 to 17, wherein the immune checkpoint inhibitor is selected from the group comprising of PD1 antagonist, PD-L1 antagonist, PD-L2 antagonist, CTLA4 antagonist or combination thereof.

Embodiment 20. The pharmaceutical composition according to embodiment 19, wherein the PD-1 antagonist is selected from the group comprising of ANA011, AUNP-12, BGB-A317, KD033, pembrolizumab, MCLA-134, mDX400, MEDI0680, muDX400, nivolumab, PDR001, PF-06801591, pidilizumab, REGN-2810, SHR-1210, STI-A1110, TSR-042, ANB011, 244C8, 388D4, TSR042 or XCE853 and the preferred one is pembrolizumab, nivolumab or pidilizumab.

Embodiment 21. The pharmaceutical composition according to embodiment 19, wherein the PD-L1 antagonist is selected from the group comprising of avelumab, BMS-936559, CA-170, durvalumab, MCLA-145, SP142, STI-A1011, STI-A1012, STI-A1010, STI-A1014, A110, KY1003 or atezolimumab and the preferred one is avelumab, durvalumab or atezolimumab.

Embodiment 22. The pharmaceutical composition according to embodiment 19, wherein the PD-L2 antagonist is selected from the group comprising of AMP-224 or rHIgM12B 7.

Embodiment 23. The pharmaceutical composition according to embodiment 19, wherein CTLA4 antagonist is selected from the group comprising of KAHR-102, AGEN1884, ABR002, KN044, tremelimumab or ipilimumab and the preferred one is tremelimumab or ipilimumab.

Embodiment 24. A therapeutic agent that binds and targets ABCB5 for use in the treatment of a tumor ameliorated by stimulation of an immune response, wherein in said treatment an immune checkpoint inhibitor, is co-administered.

Embodiment 25. A method of treating, delaying or preventing the metastasis of tumor in a subject, comprising administering to the subject an effective amount of a therapeutic agent that binds and targets ABCB5 in combination with a second therapeutic agent that binds and targets PD-1 axis, wherein the subject has been diagnosed for tumor associated with increased levels of ABCB5 and/or PD-1 axis.

Embodiment 26. A method of treating, delaying or preventing the metastasis of tumor in a subject, comprising administering to the subject an effective amount of a therapeutic agent that binds and targets ABCB5 in combination with second therapeutic agent that binds and targets CTLA4, wherein the subject has been diagnosed for tumor associated with increased levels of ABCB5 and/or CTLA4.

Embodiment 27. A combination therapy for the treatment of tumor, the said combination comprises:
(a) an effective amount of an ABCB5 inhibitor(s) and
(b) an effective amount of an immune checkpoint inhibitor(s).

Embodiment 28. A method for treating tumor comprising administering to a subject in need thereof.
(a) an effective amount of an ABCB5 inhibitor(s)and
(b) an effective amount of an immune checkpoint inhibitor(s)
to provide a combination therapy having an enhanced therapeutic effect compared to the effect of the ABCB5 inhibitor and the immune checkpoint inhibitor each administered alone.

Embodiment 29. A kit comprising
(a) a first composition comprising an ABCB5 inhibitor(s) and
(b) a second composition comprising an immune checkpoint inhibitor(s).

Embodiment 30. A method for identifying a patient diagnosed for tumor associated with increased levels of ABCB5 and/or an immune checkpoint molecule(s) having an increased probability of obtaining improved overall survival following co-administration treatment therapy with an ABCB5 inhibitor(s) and an immune checkpoint inhibitor(s).

Embodiment 31. A method of treating a subject receiving an immune checkpoint inhibitor for the treatment of tumor, the improvement comprising administering an effective amount of an ABCB5 inhibitor to the subject in conjunction with said immune checkpoint inhibitor, wherein the effect is to enhance the anti-tumor effects of said immune checkpoint inhibitor, wherein said immune checkpoint inhibitor is PD-1 antagonist, PD-L1 antagonist, PD-L2 antagonist, CTLA4 antagonist and combination thereof.

Embodiment 32. A method of enhancing IL-2 production in a subject having a tumor, comprising administering an effective amount of (a) an ABCB5 inhibitor(s) and (b) an immune checkpoint inhibitor(s) to a subject having tumor, wherein the combination of the ABCB5 inhibitor and the immune checkpoint inhibitor provide a synergistic increase in IL-2 production.

Embodiment 33. The method according to embodiment 25, wherein the PD1 axis is selected from the group consisting of a PD-1, a PD-L1 and a PD-L2.

Embodiment 34. The method according to embodiments 24 to 26, wherein the therapeutic agent that binds and targets ABCB5 is an ABCB5 inhibitor.

Embodiment 35. The method according to embodiments 27 to 32, wherein the ABCB5 inhibitor includes anti-ABCB5 antibody including anti-ABCB5 nanobody or small molecule targeting ABCB5 and the preferred one is an anti-ABCB5 antibody which is a monoclonal that binds and targets the amino acid residues corresponding to 481-674 of the human ABCB5.

Embodiment 36. The method according to embodiments 25, wherein the therapeutic agent that binds and targets PD1 axis is PD1 antagonist, PD-L1 antagonist, PD-L2 antagonist and combination thereof.

Embodiment 37. The method according to embodiment 26, wherein therapeutic agent that binds and targets CTLA4 is CTLA4 antagonist.

Embodiment 38. The method according to embodiments 24 and 27 to 32, wherein the immune checkpoint inhibitor is selected from the group consisting of PD1 axis is PD1 antagonist, PD-L1 antagonist, PD-L2 antagonist, CTLA4 antagonist and combination thereof.

Embodiment 39. The method according to embodiments 36 and 38, wherein the PD-1 antagonist is selected from the group comprising of ANA011, AUNP-12, BGB-A317, KD033, pembrolizumab, MCLA-134, mDX400, MEDI0680, muDX400, nivolumab, PDR001, PF-06801591, pidilizumab, REGN-2810, SHR-1210, STI-A1110, TSR-042, ANB011, 244C8, 388D4, TSR042 or XCE853 and the preferred one is pembrolizumab, nivolumab or pidilizumab.

Embodiment 40. The method according to embodiments 36 and 38, wherein the PD-L1 antagonist is selected from the group comprising of avelumab, BMS-936559, CA-170, durvalumab, MCLA-145, SP142, STI-A1011, STI-A1012, STI-A1010, STI-A1014, A110, KY1003 or atezolimumab and the preferred one is avelumab, durvalumab or atezolimumab.

Embodiment 41. The method according to embodiments 36 and 38, wherein the PD-L2 antagonist is selected from the group comprising of AMP-224 or rHIgM12B7.

Embodiment 42. The method according to embodiments 37 and 38, wherein CTLA4 antagonist is selected from the group comprising of KAHR-102, AGEN1884, ABR002, KN044, tremelimumab or ipilimumab and the preferred one is tremelimumab or ipilimumab.

Embodiment 43. The method according to the preceding embodiments, wherein the combination of the ABCB5 inhibitor and the immune checkpoint inhibitor is administered or contacted concurrently with, prior to, or subsequent to, the immune checkpoint inhibitor.

Embodiment 44. The method according to any of the preceding embodiments, wherein the immune checkpoint inhibitor is administered at a dose from about 0.01 to 30 mg/kg, preferably 0.1 to 20 mg/kg, more preferably 1 to 10 mg/kg.

Embodiment 45. The method according to any of the preceding embodiments, wherein the ABCB5 inhibitor is administered at a dose from about 0.1 mg/kg to 20 mg/kg, preferably 0.50 mg/kg to 10 mg/kg, more preferably 1 mg/kg to 5 mg/kg.

Embodiment 46. An anti-ABCB5 antibody or antigen-binding fragment(s) thereof, having at least one region that binds to epitope corresponding to amino acid residues 481-674 of the human ABCB5, wherein the anti-ABCB5 antibody or antigen binding fragment(s) competitively inhibits the binding of antibody having at least one region that binds to epitope corresponding to amino acid residues 1-30 of the human ABCB5 to ABCB5.

Embodiment 47. A method of reducing growth, survival, or viability, or all, of a cancer cell, comprising contacting the cell with an ABCB5 inhibitor and an immune checkpoint inhibitor, wherein:
(i) the ABCB5 inhibitor is selected from a group comprises of an antibody including nanobody, or small molecule and the preferred one is monoclonal antibody or nanobody.
(ii) the immune checkpoint inhibitor is selected from a group comprises of a PD-1 antagonist, PD-L1 antagonist, PD-L2 antagonist, CTLA4 antagonist and combination thereof
thereby reducing the growth, survival, or viability of the cancer cell.

Embodiment 48. An ABCB5 inhibitor in combination with PD-1 antagonist, wherein the PD-1 antagonist is selected from the group comprising of ANA011, AUNP-12, BGB-A317, KD033, pembrolizumab, MCLA-134, mDX400, MEDI0680, muDX400, nivolumab, PDR001, PF-06801591, pidilizumab, REGN-2810, SHR-1210, STI-A1110, TSR-042, ANB011, 244C8, 388D4, TSR042 or XCE853 and the preferred one is pembrolizumab, nivolumab or pidilizumab.

Embodiment 49. An ABCB5 inhibitor in combination with PD-L1 antagonist, wherein the PD-L1 antagonist is selected from the group comprising of avelumab, BMS-936559, CA-170, durvalumab, MCLA-145, SP142, STI-A1011, STI-A1012, STI-A1010, STI-A1014, A110, KY1003 or atezolimumab and the preferred one is avelumab, durvalumab or atezolimumab.

Embodiment 50. An ABCB5 inhibitor in combination with PD-L2 antagonist, wherein the PD-L2 antagonist is selected from the group comprising of AMP-224 or rHIgM12B7.

Embodiment 51. An ABCB5 inhibitor in combination with CTLA4 antagonist, wherein the CTLA4 antagonist is selected from the group comprising of KAHR-102, AGEN1884, ABR002, KN044, tremelimumab or ipilimumab and the preferred one is tremelimumab and ipilimumab.

Embodiment 52. A combination of ABCB5 inhibitor and PD-1 antagonist for use in the treatment of cancer.

Embodiment 53. A combination of ABCB5 inhibitor and PD-L1 antagonist for use in the treatment of cancer.

Embodiment 54. A combination of ABCB5 inhibitor and PD-L2 antagonist for use in the treatment of cancer.

Embodiment 55. A combination of ABCB5 inhibitor and CTLA4 antagonist for use in the treatment of cancer.

### Proposed Combinations of the Present Invention:

In one of the embodiments, an ABCB5 inhibitor (for example anti-ABCB5 antibody) is used in combination of an immune checkpoint inhibitor (for example PD-1 antagonist or PD-L1 antagonist or PD-L2 antagonist or CTLA4 antagonist) for the treatment of a solid tumor or cancer.

In one of the embodiments, an ABCB5 inhibitor (for example anti-ABCB5 antibody) is used in combination of an immune checkpoint inhibitor (for example PD-1 antagonist or PD-L1 antagonist or PD-L2 antagonist or CTLA4 antagonist) for the treatment of a hematological cancer.

In one of the embodiments, an ABCB5 inhibitor (for example anti-ABCB5 antibody) is used in combination of nivolumab, pembrolizumab, avelumab or ipilimumab for the treatment of the solid tumor or hematological cancer.

In one of the embodiments, anti-ABCB5 antibody which is a monoclonal that binds and targets the amino acid residues corresponding to 481-674 of the human ABCB5 is used in combination with an immune checkpoint inhibitor (for example PD-1 antagonist or PD-L1 antagonist or PD-L2 antagonist or CTLA4 antagonist) for the treatment of the solid tumor or hematological cancer.

In one of the embodiments, an ABCB5 Inhibitor (for example anti-ABCB5 antibody) is used in combination of an immune checkpoint inhibitor (for example PD-1 antagonist or PD-L1 antagonist or PD-L2 antagonist or CTLA4 antagonist) for the treatment of the solid tumor (such as melanoma, metastatic melanoma, oral squamous cell carcinoma, breast cancer, colorectal cancer, glioblastoma, hepatocellular carcinoma) or hematological cancer (leukemia, lymphoma, a lymphocytic leukemia, non-Hodgkin's lymphoma, Hodgkin's lymphoma, an anaplastic large-cell lymphoma, myeloid leukemia, multiple myeloma, acute lymphoblastic leukemia, chronic myeloid leukemia, acute myeloid leukemia).

In one of the embodiments, anti-ABCB5 antibody which is a monoclonal that binds and targets the amino acid residues corresponding to 481-674 of the human ABCB5 is used in combination of an immune checkpoint inhibitor (for example nivolumab, pembrolizumab, avelumab or ipilimumab) for the treatment of the solid tumor (such as melanoma, metastatic melanoma, oral squamous cell carcinoma, breast cancer, colorectal cancer, glioblastoma, hepatocellular carcinoma) or hematological cancer (leukemia, lymphoma, a lymphocytic leukemia, non-Hodgkin's lymphoma, Hodgkin's lymphoma, an anaplastic large-cell lymphoma, myeloid leukemia, multiple myeloma, acute lymphoblastic leukemia, chronic myeloid leukemia, acute myeloid leukemia).

In one of the embodiments, anti-ABCB5 antibody which is a monoclonal that binds and targets the amino acid residues corresponding to 481-674 of the human ABCB5 is used in combination of an immune checkpoint inhibitor (for example nivolumab, pembrolizumab, avelumab or ipilimumab) for the treatment of the melanoma, non-small cell lung cancer, renal cancer, Hodgkin's disease, unresectable or metastatic melanoma, gastric cancer, oesophageal cancer, urogenital cancer, hepatocellular carcinoma, glioblastoma, head and neck cancer, small cell lung cancer, breast cancer, colorectal cancer or multiple myeloma.

In one embodiments, one of nivolumab, pembrolizumab, avelumab or ipilimumab is used in combination with anti-ABCB5 antibody which is a monoclonal that binds and targets the amino acid residues corresponding to 481-674 of the human ABCB5 to treat a cancer or disorder described herein.

### EXAMPLES

### Example 1

Evaluation of anti-ABCB5 Antibody in combination with PD1 antagonist on IL-2 Secretion in cultures of human PBMCs & Melanoma Cell Line

Materials and methods: ABCB5 positive and negative cell lines SKMEL-28 or WM-266-4 respectively were purchased from the American Type Culture Collection (ATCC). RPMI 1640 media, 100 mM L-glutamine, 100 units/ml each penicillin and streptomycin were procured from Invitrogen, while heat inactivated FBS and trypsin were purchased from SIGMA. LPS and PHA were brought from SIGMA. Anti-ABCB5 antibody and PD-1 antagonist were purchased from Novus and BPS Biosciences respectively. The 96-well flat bottom plate from Nunc was used in the assay.

ABCB5 melanoma cells (SKMEL-28 or WM-266-4) were selected and cultured in the defined culture media and conditions. Human PBMCs were collected from the healthy volunteers and used for the activation studies. Total cells were counted and re-suspended in complete RPMI-1640 media followed by the titration of cell densities for PBMCs and melanoma cells. The cell suspensions were added to each well of the 96-well flat bottom plate and placed in a humidified 37° C., 5% CO₂ incubator under varying concentrations of anti-ABCB5 along with an optimized concentration of LPS or PHA. At the optimal incubation time, the culture supernatants were collected to estimate IL-2 secretion by ELISA using the kit manufacturer's protocol (R&D systems).

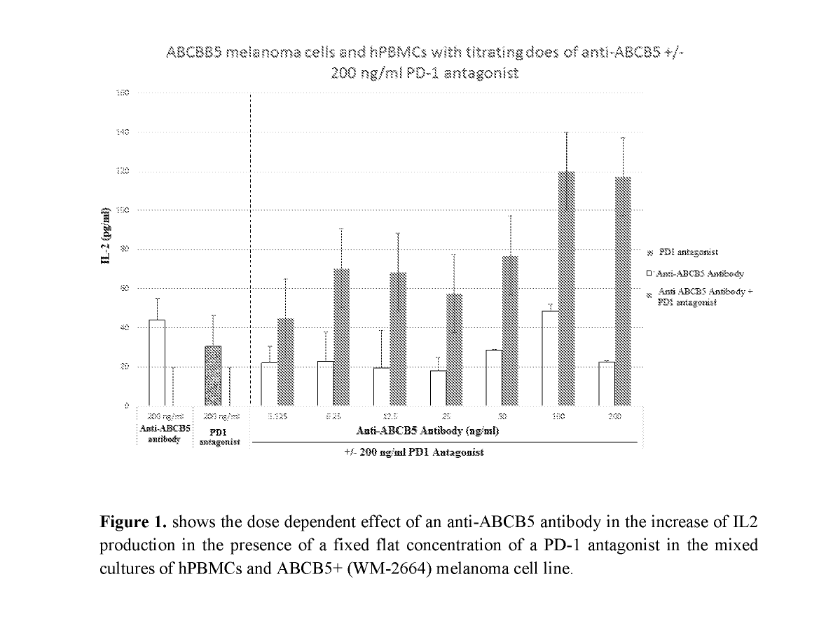

Conclusion: Anti-ABCB5 antibody at concentrations ranging from 3.13 to 200 ng/ml showed a dose dependent increase on the IL-2 production in presence of a flat fixed concentration (200 ng/ml) of PD-1 antagonist after 72 hours of incubation at an optimal target to effector cell (ABCB5+ melanoma cells to hPBMCs) as illustrated in FIG. 1. As such in the absence of either of the agents there was a background release of IL-2 (+/−20 pg/ml) in the mixed cultures of ABCB5+ melanoma cell and hPBMCs.

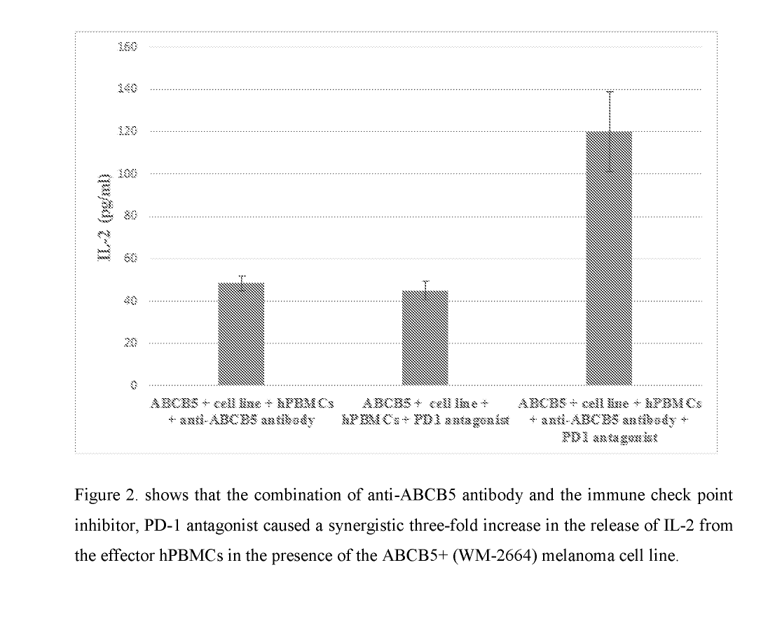

Moreover, the combination of anti-ABCB5 antibody and PD-1 antagonist at concentrations of 200 ng/ml showed a synergistic threefold increase in IL-2 production in mixed cultures of ABCB5+ melanoma cell line (WM-2664) and hPBMCs as illustrated in FIG. 2.

Thus it can be concluded that a combination of an anti-ABCB5 antibody and an immune checkpoint inhibitor produce a synergistic effect.

### Definitions

The term “subject” includes any organism, preferably an animal, more preferably a mammal (e.g., rat, mouse, dog, cat, rabbit) and most preferably a human.

As used herein the term “cancer” can be used interchangeably with “tumor”. The term “cancer” refers to the cancers of wide variety of types, including both solid tumors and non-solid tumors such as leukemia and lymphoma. Carcinomas, sarcomas, myelomas, lymphomas, and leukemia can all be treated using the present invention, including those cancers which have a mixed type. The present invention can be used to treat either malignant or benign tumors or solid or leukemia. In some embodiments, the cancer is urogenital cancers (such as prostate cancer, renal cell cancer, bladder cancer), thyroid cancer, testicular cancer, vulvar cancer, Wilm's tumor, rhabdomyosarcoma, retinoblastoma, hormone sensitive or hormone refractory prostate cancer, gynecological cancers (such as ovarian cancer, cervical cancer, endometrial cancer, uterine cancer), lung cancer, non-small cell lung cancer, small cell lung cancer, gastrointestinal stromal cancers, gastrointestinal cancers (such as non-metastatic or metastatic colorectal cancers, pancreatic cancer, gastric cancer, oesophageal cancer, hepatocellular cancer, cholangiocellular cancer), head and neck cancer (such as head and neck squamous cell cancer), malignant glioblastoma, malignant mesothelioma, non-metastatic or metastatic breast cancer (such as hormone refractory metastatic breast cancer, triple negative breast cancer), malignant melanoma, melanoma, metastatic melanoma, merkel cell carcinoma or bone and soft tissue sarcomas, oral squamous cell carcinoma, glioblastoma, brain cancer, osteosarcoma, neuroblastoma, advanced metastatic, an inflammatory myofibroblastic tumor (IMT), cholangiocarcinoma, cystadenocarcionoma, ameloblastoma, chondrosarcoma, dermatofibrosarcoma, ganglioglioma, leiomyosarcoma, medulloblastomma, osteoblastoma and inoperable non-inflammatory locally advanced disease and the like. The most preferred cancer is solid tumor (such as melanoma, metastatic melanoma, oral squamous cell carcinoma, breast cancer, colorectal cancer, glioblastoma, hepatocellular carcinoma) or hematological cancer (leukemia, lymphoma, a lymphocytic leukemia, non-Hodgkin's lymphoma, Hodgkin's lymphoma, an anaplastic large-cell lymphoma, myeloid leukemia, multiple myeloma, acute lymphoblastic leukemia, chronic myeloid leukemia, acute myeloid leukemia). The cancer may be at an early, intermediate, late stage or metastatic cancer.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The term “Treating” within the context of the present invention, means an alleviation of symptoms associated with a disorder or disease, or halt of further progression or worsening of those symptoms, or prevention or prophylaxis of the disease or disorder. For example, within the context of treating patients in relation to the ABCB5 inhibitor and an immune checkpoint inhibitor, successful treatment may include a reduction in tumor adhesion and anchorage; an alleviation of symptoms related to a cancerous growth or tumor, or proliferation of diseased tissue; a halting in the progression of a disease such as cancer or in the growth of cancerous cells. Treatment may also include administering the pharmaceutical formulations of an ABCB5 inhibitor in combination with an immune checkpoint inhibitor. It may be administered before, during, or after surgical procedure and/or radiation therapy. According to this invention, an ABCB5 inhibitor and an immune checkpoint inhibitor can be co-administered into a human subject, the daily dosage will normally be determined by the prescribing physician with the dosage generally varying according to the age, weight, and response of the individual patient, as well as the severity of the patient's symptoms.

When introducing elements disclosed herein, the articles “a”, “an”, “the”, and “said” are intended to mean that there are one or more of the elements.

As used herein the term “effective amount” can be used interchangeably with “therapeutically effective dose,” or “therapeutically effective amount,” and it refers to an amount sufficient to produce the desired effect.

As used herein “pharmaceutical acceptable carrier” refers to a carrier medium which does not interfere with the effectiveness of the biological activity of the active ingredients and which is not toxic to the patient or subject. As used herein the term “carrier” can be used interchangeably with “adjuvant”.

The term “pharmaceutical composition” as used in accordance with the present invention relates to compositions that can be formulated in any conventional manner using one or more pharmaceutically acceptable carriers or adjuvants.

The term “monoclonal antibody” or “monoclonal antibody composition,” as used herein, refers to an antibody or a composition of antibodies that displays a single binding specificity and affinity for a particular epitope. Accordingly, the term “human monoclonal antibody” or “monoclonal antibody composition” refers to an antibody or a composition of antibodies which displays a single binding specificity and which has variable and optional constant regions derived from human germline immunoglobulin sequences. In one embodiment, human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell. In another embodiment, the monoclonal antibody can also be produced by recombinant technology. The term “epitope” or “antigenic determinant” refers to a site on an antigen to which an immunoglobulin or antibody specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)).

A nanobody (Nb) is the smallest functional fragment or single variable domain (VHH) of a naturally occurring single-chain antibody and is known to the person skilled in the art. They are derived from heavy chain only antibodies, seen in camelids (Hamers-Casterman et al. 1993; Desmyter et al. 1996). In the family of “camelids” immunoglobulins devoid of light polypeptide chains are found. “Camelids” comprise old world camelids (Camelus bactrianus and Camelus dromedarius) and new world camelids (for example Lama paccos, Lama glama, Lama guanicoe and Lama vicugna). Said single variable domain heavy chain antibody is herein designated as a Nanobody or a VHH antibody. Nanobody™ Nanobodies™ and Nanoclone™ are trademarks of Ablynx NV (Belgium).

The term “polyclonal antibody” refers to preparations that include different antibodies directed against different determinants (“epitopes”).

As used herein, the term “synergy” refers generally to obtaining a combined effect that is greater than the sum of two separate effects. As used herein, the terms “therapeutic synergy”, and “synergistic effect,” when placed in a therapeutic context, refer to a phenomenon where treatment of patients with a combination of therapeutic agents (e.g., ABCB5 inhibitor in combination with anti-PD1 or anti-PD L1 or anti CTLA4) manifests a therapeutically superior outcome to the outcome achieved by each individual constituent of the combination used at its optimum dose (see, e.g., T. H. Corbett et al., 1982, Cancer Treatment Reports, 66, 1187). In this context a therapeutically superior outcome is one in which the patients either a) exhibit fewer incidences of adverse events while receiving a therapeutic benefit that is equal to or greater than that where individual constituents of the combination are each administered as monotherapy at the same dose as in the combination, or b) do not exhibit dose-limiting toxicities while receiving therapeutic benefit that is greater than that of treatment with each individual constituent of the combination when each constituent is administered in at the same doses in the combination(s) as is administered as individual components or c) both when combined produces enhanced effects as compared to when given alone, for example increase in IL-2 release. In xenograft models, a combination, used at its maximum tolerated dose, in which each of the constituents will be present at a dose generally not exceeding its individual maximum tolerated dose, manifests therapeutic synergy when decrease in tumor growth achieved by administration of the combination is greater than the value of the decrease in tumor growth of the best constituent when the constituent is administered alone.

As used herein, the terms “therapeutic agent” and “therapeutic agents” refer to an agent that can be used in the prevention, treatment, management, or amelioration of a disorder associated with increased levels of ABCB5 or immune checkpoint molecule (e.g., cancer) or one or more symptoms thereof. In certain embodiments, the term “therapeutic agent” refers to an antibody or nanobody or small molecule that binds and targets ABCB5 or immune checkpoint molecule.

1. (canceled)

2. A method of enhancing an immune response in a subject, comprising administering to the subject an effective amount of a first therapeutic agent that binds and targets ABCB5 in combination with a second therapeutic agent that binds and targets an immune checkpoint molecule.

3. The method according to claim 2, wherein each of the first therapeutic agent and the second therapeutic agent is independently selected from the group consisting of an antibody including a monoclonal antibody, a polyclonal antibody, a nanobody, a small molecule, and combinations thereof, and wherein the preferred one is a monoclonal antibody and a nanobody.

4. The method according to claim 2, wherein the subject has been diagnosed as having tumor associated with increased levels of ABCB5 and/or an immune checkpoint molecule.

5. The method according to claim 2, wherein the immune checkpoint molecule is selected from the group consisting of a CTLA4 molecule, a PD-1 axis molecule, a PD-1 molecule, a PD-L1 molecule, a PD-L2 molecule, and combinations thereof.

6. The method according to claim 2, wherein the therapeutic agent that binds and targets ABCB5 is an ABCB5 inhibitor selected from the group consisting of an anti-ABCB5 antibody including an anti-ABCB5 nanobody, a small molecule targeting ABCB5, and combinations thereof.

7. The method according to claim 2, wherein the therapeutic agent that binds and targets the immune checkpoint molecule is an immune checkpoint inhibitor selected from the group consisting of a PD-1 antagonist, a PD-L1 antagonist, a PD-L2 antagonist, a CTLA4 antagonist, and combinations thereof.

8. The method according to claim 6, wherein the anti-ABCB5 antibody is a monoclonal antibody that binds and targets the amino acid residues corresponding to 481-674 of the human ABCB5.

9. The method according to claim 7, wherein the PD-1 antagonist is selected from the group consisting of ANA011, AUNP-12, BGB-A317, KD033, pembrolizumab, MCLA-134, mDX400, MEDI0680, muDX400, nivolumab, PDR001, PF-06801591, pidilizumab, REGN-2810, SHR-1210, STI-A1110, TSR-042, ANB011, 244C8, 388D4, TSR042, XCE853, and combinations thereof, and wherein the preferred one is pembrolizumab, nivolumab and pidilizumab.

10. The method according to claim 7, wherein the PD-L1 antagonist is selected from the group consisting of avelumab, BMS-936559, CA-170, durvalumab, MCLA-145, SP142, STI-A1011, STI-A1012, STI-A1010, STI-A1014, A110, KY1003, atezolimumab, and combinations thereof, and wherein the preferred one is avelumab, durvalumab and atezolimumab.

11. The method according to claim 7, wherein the PD-L2 antagonist is selected from the group consisting of AMP-224, rHIgM12B7, and combinations thereof.

12. The method according to claim 7, wherein the CTLA4 antagonist is selected from the group consisting of KAHR-102, AGEN1884, ABR002, KN044, tremelimumab, ipilimumab, and combinations thereof, and wherein the preferred one is tremelimumab and ipilimumab.

13. The method according to claim 2, wherein the subject has tumor selected from the group consisting of melanoma, metastatic melanoma, oral squamous cell carcinoma, breast cancer, colorectal cancer, glioblastoma, hepatocellular carcinoma, leukemia, lymphoma, a lymphocytic leukemia, non-Hodgkin's lymphoma, Hodgkin's lymphoma, an anaplastic large-cell lymphoma, myeloid leukemia, multiple myeloma, acute lymphoblastic leukemia, chronic myeloid leukemia, and acute myeloid leukemia.

14-27. (canceled)

28. A method for treating tumor comprising administering to a subject in need thereof
(a) an effective amount of an ABCB5 inhibitor(s) and
(b) an effective amount of an immune checkpoint inhibitor(s)
to provide a combination therapy having an enhanced therapeutic effect compared to the effect of administering the ABCB5 inhibitor or the immune checkpoint inhibitor alone.

29-31. (canceled)

32. A method of enhancing IL-2 production in a subject having a tumor, comprising administering an effective amount of (a) an ABCB5 inhibitor and (b) an immune checkpoint inhibitor to the subject, wherein a combination of the ABCB 5 inhibitor and the immune checkpoint inhibitor provides a synergistic increase in IL-2 production.

33-34. (canceled)

35. The method according to claim 28, wherein the ABCB5 inhibitor includes an anti-ABCB5 antibody including an anti-ABCB5 monoclonal antibody, an anti-ABCB5 nanobody, and a small molecule targeting ABCB5, and wherein the preferred one is an anti-ABCB5 monoclonal antibody that binds and targets the amino acid residues corresponding to 481-674 of the human ABCB5.

36-37. (canceled)

38. The method according to claim 28, wherein the immune checkpoint inhibitor is selected from the group consisting of a PD1 axis antagonist, a PD1 antagonist, a PD-L1 antagonist, a PD-L2 antagonist, a CTLA4 antagonist, and combinations thereof.

39. The method according to claim 38, wherein the PD-1 antagonist is selected from the group consisting of ANA011, AUNP-12, BGB-A317, KD033, pembrolizumab, MCLA-134, mDX400, MEDI0680, muDX400, nivolumab, PDR001, PF-06801591, pidilizumab, REGN-2810, SHR-1210, STI-A1110, TSR-042, ANB011, 244C8, 388D4, TSR042, XCE853, and combinations thereof, and wherein the preferred one is pembrolizumab, nivolumab pidilizumab, and combinations thereof.

40. The method according to claim 38, wherein the PD-L1 antagonist is selected from the group consisting of avelumab, BMS-936559, CA-170, durvalumab, MCLA-145, SP142, STI-A1011, STI-A1012, STI-A1010, STI-A1014, A110, KY1003, atezolimumab, and combinations thereof, and wherein the preferred one is avelumab, durvalumab and atezolimumab.

41. The method according to claim 38, wherein the PD-L2 antagonist is selected from the group consisting of AMP-224, rHIgM12B7, and combinations thereof.

42. The method according to claim 38, wherein the CTLA4 antagonist is selected from the group consisting of KAHR-102, AGEN1884, ABR002, KN044, tremelimumab, ipilimumab, and combinations thereof, and wherein the preferred one is tremelimumab and ipilimumab.

43. The method according to claim 28, wherein the combination of the ABCB5 inhibitor and the immune checkpoint inhibitor is administered or contacted concurrently with, prior to, or subsequent to, the immune checkpoint inhibitor.

44. The method according to claim 28, wherein the immune checkpoint inhibitor is administered at a dose from about 0.01 mg/kg to about 30 mg/kg, preferably about 0.1 mg/kg to about 20 mg/kg, more preferably about 1 mg/kg to about 10 mg/kg.

45. The method according to claim 28, wherein the ABCB5 inhibitor is administered at a dose from about 0.1 mg/kg to about 20 mg/kg, preferably about 0.50 mg/kg to about 10 mg/kg, more preferably about 1 mg/kg to about 5 mg/kg.
